(19) United States
(12) Patent Application Publication
(10) Pub. No.: US 2010/0021885 A1
(43) Pub. Date: Jan. 28, 2010

(54) REAGENT SETS AND GENE SIGNATURES FOR NON-GENOTOXIC HEPATOCARCINOGENICITY

(76) Inventors: Mark Fielden, Mountain View, CA (US); Richard John Brennan, San Jose, CA (US)

Correspondence Address: HOWREY LLP-CA, C/O IP DOCKETING DEPARTMENT, 2941 FAIRVIEW PARK DRIVE, SUITE 200, FALLS CHURCH, VA, 22042-2924, US

(21) Appl. No.: 11/856,608

(22) Filed: Sep. 17, 2007

Related U.S. Application Data
(60) Provisional application No. 60/845,751, filed on Sep. 18, 2006.

(51) Int. Cl. C12Q 1/68; C12Q 1/02

(52) U.S. Cl. 435/6; 435/29

(57) ABSTRACT

The invention discloses gene signatures for predicting onset of non-genotoxic hepatocarcinogenicity in a subject. The invention also provides methods, apparatuses and reagent sets useful for predicting non-genotoxic hepatocarcinogenicity based on expression levels of genes in specific gene signatures.

### CROSS REFERENCE TO RELATED APPLICATIONS

This application claims priority of U.S. provisional application 60/845,751, filed Sep. 18, 2006, which is hereby incorporated by reference herein.

### REFERENCE TO ELECTRONIC TABLE AND SEQUENCE LISTING FILES

This application includes Table 3 submitted electronically herewith as an ASCII formatted file named “table3.txt” which is 287 kB in size and was created Sep. 17, 2007. This application also includes a sequence listing submitted electronically herewith as an ASCII formatted filed named “sequence.txt” which is 65.2 kB in size and was created Sep. 17, 2007. These files submitted electronically herewith are referred to in the text below and each is incorporated by reference herein in its entirety.

### FIELD OF THE INVENTION

This invention relates to reagent sets and gene signatures useful for predicting the occurrence of non-genotoxic hepatocarcinogenicity in a subject. The invention also provides methods, apparatuses and kits useful for predicting occurrence of non-genotoxic hepatocarcinogenicity based on expression levels of genes in the signatures. In one embodiment, the invention provides a method for predicting whether a compound will induce non-genotoxic hepatocarcinogenicity using gene expression data from sub-acute treatments.

### BACKGROUND OF THE INVENTION

Chemical-induced liver tumors in rodents are the most frequently observed cancer in response to long term exposure to both genotoxic and non-genotoxic chemicals of commercial, therapeutic and environmental interest (see e.g., Gold et al., “Supplement to the Carcinogenic Potency Database (CPDB): results of animal bioassays published in the general literature through 1997 and by the National Toxicology Program in 1997-1998,” Toxicol Sci. 85(2):747-808 (2005); and Davies and Monro, “Marketed human pharmaceuticals reported to be tumorigenic in rodents,” J. Am. Coll. Toxicol. 14:90-107 (1995)), thus raising concern over the long term safety to humans exposed to such chemicals. Since mutation and DNA damage is believed to be the initiating event in carcinogenesis, a number of in vitro and short term in vivo tests are commonly used to identify potentially genotoxic hepatocarcinogens. However, genotoxicity does not always correlate with carcinogenicity and non-genotoxic hepatocarcinogens can be carcinogenic. As a result, the two year rodent bioassay has become the standard test for assessing carcinogenicity. Not withstanding the difficulty in extrapolating results from rodents to humans, the bioassay costs millions of dollars per compound and many years to complete. As a result, these tests are not applied until very late in development after considerable resources have already been invested, or not at all. Positive findings in these tests can often result in a delay or denial of successful registration at significantly added development costs. Furthermore, many chemicals humans are exposed to have not been adequately tested for carcinogenicity due to the resources required.

It has been proposed that a combination of mechanism-based in silico, in vitro or short term in vivo tests can replace the two year bioassay (see e.g., Cohen, “Human carcinogenic risk evaluation: an alternative approach to the two-year rodent bioassay,” Toxicol Sci. 80(2):225-9 (2004); and McDonald, “Human carcinogenic risk evaluation, part IV: assessment of human risk of cancer from chemical exposure using a global weight-of-evidence approach,” Tox Sci 82: 3-8 (2004)), however retrospective evaluations of their predictivity have indicated they are not adequate for regulatory purposes (see e.g., Jacobs, “Prediction of 2-year carcinogenicity study results for pharmaceutical products: how are we doing,” Toxicol Sci. 88(1): 18-23 (2005)) and their utility as early screening tools is questionable due to limited validation. As a result, it is doubtful whether in vitro or short term in vivo tests can adequately replace the need for the two year rodent bioassay. Medium term bioassays in rats and transgenic mice have been developed as alternatives to long term testing (Ito et al., “A medium-term rat liver bioassay for rapid in vivo detection of carcinogenic potential of chemicals,” Cancer Sci. 94(1):3-8 (2003); Cohen et al. “Alternative models for carcinogenicity testing,” Toxicol Sci. 64(1): 14-9 (2001)), however their format and expense do not facilitate routine screening. Furthermore, they not provide information to begin to assess mode of action (MOA).

The development of methods to predict the future onset of non-genotoxic hepatocarcinogenicity and gain a greater understanding of the underlying mechanism, would facilitate the development more reliable clinical diagnostics and safer therapeutic drugs. In addition, improved preclinical markers for non-genotoxic hepatocarcinogenicity would dramatically reduce the time, cost, and amount of compound required in order to prioritize and select lead candidates for progression through drug development. Thus, there is a need in the art for a pragmatic approach that applies short term screening methods and provides an early warning of potential hazard as well as MOA information that can be used to guide decision making and/or prioritization of chemicals for further development or evaluation.

### SUMMARY OF THE INVENTION

The present invention provides a gene expression based approach to identify chemical-induced hepatic transcripts that are predictive of long-term hepatocarcinogenicity. More specifically, the present invention provides methods, reagent sets, gene sets, and associated apparatuses and kits that allow one to determine the onset of non-genotoxic hepatocarcinogenicity by measuring gene expression levels. Additionally, the multi-gene biomarkers, or signatures provided herein, when compared to reference compounds of known mechanism may be used to identify testable hypotheses of MOA.

The present invention provides a plurality of specific gene signatures (i.e., linear classifiers) for non-genotoxic hepatocarcinogenicity. In one embodiment, the gene signature for non-genotoxic hepatocarcinogenicity comprises the 37 genes, their associated weights, and the bias value listed in Table 4.

The methods for deriving gene signatures disclosed herein are capable of providing a plurality of gene signature for non-genotoxic hepatocarcinogenicity capable of different levels of performance for classification. In one embodiment, the invention provides a gene signature wherein the signature classifies each of the compounds listed in Table 2 according to its label as in-class or not in-class for non-genotoxic hepatocarcinogenicity.

In one embodiment, the invention provides a gene signature for non-genotoxic hepatocarcinogenicity that is capable of classifying a true label set with a log odds ratio at least 2 standard deviations greater than its performance classifying a random label set. In a preferred embodiment, the invention provides a gene signature for non-genotoxic hepatocarcinogenicity is capable of performing with a training log odds ratio of greater than or equal to 2.00, 2.25, 2.50, 2.75, 2.85, or 2.90 or greater.

Additionally, for each of the plurality of gene signatures for non-genotoxic hepatocarcinogenicity provided herein, the present invention also provides reagent sets and kits comprising polynucleotides and/or polypeptides that represent the plurality of genes selected from the gene signature. In one embodiment, the present invention provides a reagent set comprising a set of polynucleotides and/or polypeptides representing at least the 37 genes listed in Table 4.

In one embodiment, the plurality of genes in the reagent set are selected from the variables of a linear classifier capable of classifying non-genotoxic hepatocarcinogenicity with a training log odds ratio of greater than or equal to 2.00, 2.25, 2.50, 2.75, 2.90 or greater. In one preferred embodiment, the plurality of genes is the set of genes in Table 4. In one embodiment, the reagents are polynucleotide probes capable of hybridizing to a plurality of genes selected from those listed in Table 4, and in a preferred embodiment, the polynucleotide probes are labeled.

In another embodiment, the reagents are primers for amplification of the plurality of genes. In one embodiment the reagents are polypeptides encoded by a plurality of genes selected from those listed in Table 4. Preferably the reagents are polypeptides that bind to a plurality proteins encoded by a plurality of genes selected from those listed in Table 4. In one preferred embodiment, the reagent set comprises secreted proteins encoded by genes listed in Table 4.

In one embodiment, the reagent sets consist essentially of polynucleotides or polypeptides representing the plurality of genes from Table 4. Further, the invention comprises kits comprising the reagent sets as components. In one embodiment, the reagent set is packaged in a single container consisting essentially of polynucleotides or polypeptides representing the plurality of genes from Table 4.

The gene signatures (and associated and reagent sets) for non-genotoxic hepatocarcinogenicity provided by the present invention are useful for testing whether non-genotoxic hepatocarcinogenicity will occur in a test subject. Thus, the present invention provides methods for testing or monitoring hepatocarcinogenicity, and devices for carrying out these methods.

Thus, in one embodiment, the present invention provides a method for testing whether a compound will induce non-genotoxic hepatocarcinogenicity in a test subject, the method comprising: administering a dose of a compound to at least one test subject; after a selected time period, obtaining a biological sample from the at least one test subject; measuring the expression levels in the biological sample of genes selected from a gene signature for non-genotoxic hepatocarcinogenicity; and determining whether the sample is in the positive class for non-genotoxic hepatocarcinogenicity using the gene signature comprising at least the plurality of genes for which the expression levels are measured. In one embodiment, the gene signature used in the method comprises the 37 genes, their associated weights, and the bias value listed in Table 4. In one embodiment, the method is carried out wherein the test subject is a mammal selected from the group consisting of a human, cat, dog, monkey, mouse, pig, rabbit, and rat. In one preferred embodiment the test subject is a rat. In one embodiment, the biological sample comprises kidney tissue. In one embodiment, the method is carried out wherein the test compound is administered to the subject intravenously (IV), orally (PO, per os), or intraperitoneally (IP). In one embodiment, the method is carried out wherein the dose administered does not cause histological or clinical evidence of non-genotoxic hepatocarcinogenicity at about 5 days, about 7 days, about 14 days, or even about 21 days. In one embodiment, the method is carried out wherein the expression levels are measured as log₁₀ ratios of compound-treated biological sample to a compound-untreated biological sample. In one embodiment, the method of the invention is carried out wherein the classifier is a linear classifier. In alternative embodiments, the classifier may be a non-linear classifier. In one embodiment, the method is carried out wherein the selected period of time is about 5 days or fewer, 7 days or fewer, 14 days or fewer, or even 21 days or fewer. In one embodiment of the method, the selected period of time is at least about 28 days.

In one embodiment the present invention provides a method for predicting non-genotoxic hepatocarcinogenicity in an individual comprising: obtaining a biological sample from the individual after short-term treatment with compound; measuring the expression levels in the biological sample of genes selected from a gene signature for non-genotoxic hepatocarcinogenicity; and determining whether the sample is in the positive class for non-genotoxic hepatocarcinogenicity using the gene signature comprising at least the plurality of genes for which the expression levels are measured; wherein a sample in the positive class indicates that the individual will have non-genotoxic hepatocarcinogenicity following sub-chronic treatment with compound. In one embodiment, the gene signature used in the method comprises the 37 genes, their associated weights, and the bias value listed in Table 4. In one preferred embodiment, the method for predicting non-genotoxic hepatocarcinogenicity is carried out wherein the genes encode secreted proteins. In a preferred embodiment, the individual is a mammal, and preferably a rat. In another preferred embodiment, the biological sample is selected from blood, urine, hair or saliva. In another preferred embodiment of the method, the expression log₁₀ ratio is measured using an array of polynucleotides.

In another embodiment, the invention provides a method for monitoring treatment of an individual for non-genotoxic hepatocarcinogenicity, or with a compound suspected of causing non-genotoxic hepatocarcinogenicity, said method comprising: obtaining a biological sample from the individual after short-term treatment with compound; measuring the expression levels in the biological sample of genes selected from a gene signature for non-genotoxic hepatocarcinogenicity; and determining whether the sample is in the positive class for non-genotoxic hepatocarcinogenicity using the gene signature comprising at least the plurality of genes for which the expression levels are measured; wherein a sample in the positive class indicates that the individual will have non-genotoxic hepatocarcinogenicity. In one embodiment, the gene signature used in the method comprises the 37 genes, their associated weights, and the bias value listed in Table 4. In a preferred embodiment, the individual is a mammal, and preferably a rat. In another preferred embodiment, the biological sample is selected from blood, urine, hair or saliva. In another preferred embodiment of the method, the expression log₁₀ ratio is measured using an array of polynucleotides.

In one embodiment, the invention provides a non-genotoxic hepatocarcinogenicity “necessary set” of genes mined from a chemogenomic dataset. These genes are information-rich with respect to classifying biological samples for onset of non-genotoxic hepatocarcinogenicity, even at sub-acute doses and time points of 5 days or earlier, where clinical and histopathological evidence of non-genotoxic hepatocarcinogenicity are not manifested. Further, the necessary set for non-genotoxic hepatocarcinogenicity classification has the functional characteristic of reviving the performance of a fully depleted set of genes (for classifying non-genotoxic hepatocarcinogenicity) by supplementation with random selections of 20%, 15%, 10%, 5%, or even fewer of the genes from the necessary set. In addition, the invention discloses that selections from the necessary set made based on percentage impact of the selected genes may be used to generate high-performing linear classifiers for non-genotoxic hepatocarcinogenicity.

In one embodiment, the invention provides a reagent set comprising a plurality of polynucleotides or polypeptides representing a plurality of genes selected from those listed in the necessary set. In one embodiment, the reagent set comprises a plurality of genes selected from those in the necessary set, the plurality of genes having at least at least 1, 2, 4, 8, 16, 32, or 64% of the total impact of all of the genes in the necessary set. In one embodiment, the reagent set represents as few genes as possible from the necessary set while maximizing percentage of total impact. In one embodiment, the reagent set includes fewer than 1000, 500, 400, 300, 200, 100, 50, 20, 10, or even 8, polynucleotides or polypeptides representing the plurality of genes from the necessary set.

In one embodiment, the invention provides a reagent set for classifying non-genotoxic hepatocarcinogenicity comprising a set of polynucleotides or polypeptides representing a plurality of genes selected from the necessary set, wherein the addition of a random selection of at least 10% of said plurality of genes to the fully depleted set for the non-genotoxic hepatocarcinogenicity classification question increases the average logodds ratio of the linear classifiers generated by the depleted set by at least 3-fold. In another embodiment, the reagent set includes at least 20% of said plurality of genes to the fully depleted set for the non-genotoxic hepatocarcinogenicity classification question increases the average logodds ratio of the linear classifiers generated by the depleted set by at least 2-fold.

The present invention also provides an apparatus for predicting whether non-genotoxic hepatocarcinogenicity will occur in a test subject comprising a reagent set as described above. In preferred embodiments, the apparatus comprises a device with reagents for detecting polynucleotides, wherein the reagents comprise or consist essentially of a reagent set for testing whether non-genotoxic hepatocarcinogenicity will occur in a test subject as described above.

In one embodiment, the apparatus comprises at least a plurality of polynucleotides or polypeptides representing a plurality of genes selected from those in the necessary set. In one embodiment the apparatus comprises a plurality of genes includes at least 4 genes selected from those in the necessary set, the four genes having at least 2% of the total impact of the genes in the necessary set.

In one embodiment, the apparatus comprises polynucleotide probes capable of hybridizing to a plurality of genes selected from those listed in the necessary set. In preferred embodiments, the apparatus comprises a plurality of polynucleotide probes bound to one or more solid surfaces. In one embodiment, the plurality of probes is bound to a single solid surface in an array. Alternatively, the plurality of probes is bound to the solid surface on a plurality of beads. In another preferred embodiment, the apparatus comprises polypeptides encoded by a plurality of genes selected from those listed in the necessary set. In one preferred embodiment, the polypeptides are secreted proteins encoded by genes listed in the necessary set.

### BRIEF DESCRIPTION OF THE DRAWINGS

FIG. 1 depicts the 37 gene non-genotoxic hepatocarcinogenicity signature derived according to the method of Example 3, and visualizes the average expression log₁₀ ratio in the compound training positive class using a “heat map” depiction.

### DETAILED DESCRIPTION OF THE INVENTION

### I. Overview

The present invention provides methods for predicting onset of non-genotoxic hepatocarcinogenicity following sub-acute or short-term compound treatment using gene expression data. The invention provides a specific 37 gene signature allows gene expression data to be used to identify the ability of a compound treatment to induce onset of non-genotoxic hepatocarcinogenicity before the actual histological or clinical indication of the toxicity. Further, the invention provides reagent sets and diagnostic devices comprising the disclosed gene signature that may be used to deduce compound toxicity using short term studies, and avoiding lengthy and costly long term studies. The invention also provides methods of using the gene signature, and associated devices and apparatus, for diagnosis and therapeutic monitoring of non-genotoxic hepatocarcinogenicity.

### II. Definitions

“Multivariate dataset” as used herein, refers to any dataset comprising a plurality of different variables including but not limited to chemogenomic datasets comprising log₁₀ ratios from differential gene expression experiments, such as those carried out on polynucleotide microarrays, or multiple protein binding affinities measured using a protein chip. Other examples of multivariate data include assemblies of data from a plurality of standard toxicological or pharmacological assays (e.g., blood analytes measured using enzymatic assays, antibody based ELISA or other detection techniques).

“Variable” as used herein, refers to any value that may vary. For example, variables may include relative or absolute amounts of biological molecules, such as mRNA or proteins, or other biological metabolites. Variables may also include dosing amounts of test compounds.

“Signature,” “drug signature,” “classifier” or “linear classifier” as used herein, refers to a linear function comprising a combination of variables, weighting factors, and other constants that provides a unique value or function capable of answering a classification question and whose cross-validated performance for answering a specific classification question is greater than an arbitrary threshold (e.g., a log odds ratio≧4.0). The “classification question” may be of any type susceptible to yielding a yes or no answer (e.g., “Is the unknown a member of the class or does it belong with everything else outside the class?”). “Linear classifiers” refers to classifiers comprising a first order function of a set of variables, for example, a summation of a weighted set of gene expression log₁₀ ratios. A valid classifier is defined as a classifier capable of achieving a performance for its classification task at or above a selected threshold value. For example, a log odds ratio≧4.00 represents a preferred threshold of the present invention. Higher or lower threshold values may be selected depending of the specific classification task.

“Weighting factor” (or “weight”) as used herein, refers to a value used by an algorithm in combination with a variable in order to adjust the contribution of the variable.

“Impact factor” or “Impact” as used herein in the context of classifiers or signatures refers to the product of the weighting factor by the average value of the variable of interest. For example, where gene expression log ratios are the variables, the product of the gene's weighting factor and the gene's measured expression log 10 ratio yields the gene's impact. The sum of the impacts of all of the variables (e.g., genes) in a set yields the “total impact” for that set.

“Scalar product” (or “Signature score”) as used herein refers to the sum of impacts for all genes in a signature less the bias for that signature. A positive scalar product for a sample indicates that it is positive for (i.e., a member of) the classification that is determined by the classifier or signature.

“Sufficient set” as used herein is a set of variables (e.g., genes, weights, bias factors) whose cross-validated performance for answering a specific classification question is greater than an arbitrary threshold (e.g., a log odds ratio≧4.0).

“Necessary set” as used herein is a set of variables whose removal from the full set of all variables results in a depleted set whose performance for answering a specific classification question does not rise above an arbitrarily defined minimum level (e.g., log odds ratio≧4.00).

“Log odds ratio” or “LOR” is used herein to summarize the performance of classifiers or signatures. LOR is defined generally as the natural log of the ratio of the odds of predicting a subject to be positive when it is positive, versus the odds of predicting a subject to be positive when it is negative. LOR is estimated herein using a set of training or test cross-validation partitions according to the following equation,

$$L O R = ln \frac{( {\sum }_{i = 1}^{c} {TP}_{i} + 0.5 ) * ( {\sum }_{i = 1}^{c} {TN}_{i} + 0.5 )}{( {\sum }_{i = 1}^{c} {FP}_{i} + 0.5 ) * ( {\sum }_{i = 1}^{c} {FN}_{i} + 0.5 )}$$

where c (typically c=40 as described herein) equals the number of partitions, and TPᵢ, TNᵢ, FPᵢ, and FNᵢ represent the number of true positive, true negative, false positive, and false negative occurrences in the test cases of the i^{th} partition, respectively.

“Array” as used herein, refers to a set of different biological molecules (e.g., polynucleotides, peptides, carbohydrates, etc.). An array may be immobilized in or on one or more solid substrates (e.g., glass slides, beads, or gels) or may be a collection of different molecules in solution (e.g., a set of PCR primers). An array may include a plurality of biological polymers of a single class (e.g., polynucleotides) or a mixture of different classes of biopolymers (e.g., an array including both proteins and nucleic acids immobilized on a single substrate).

“Array data” as used herein refers to any set of constants and/or variables that may be observed, measured or otherwise derived from an experiment using an array, including but not limited to: fluorescence (or other signaling moiety) intensity ratios, binding affinities, hybridization stringency, temperature, buffer concentrations.

“Proteomic data” as used herein refers to any set of constants and/or variables that may be observed, measured or otherwise derived from an experiment involving a plurality of mRNA translation products (e.g., proteins, peptides, etc) and/or small molecular weight metabolites or exhaled gases associated with these translation products.

“Sample” as used herein refers to any biological sample used to assay a compound or treatment (e.g., cell culture, tissue culture, or organism such as an animal or human).

“Ortholog” as used herein refers to at least two genes found in different species that are related by vertical descent from a common ancestor and encode proteins with the same function. Over 13000 human orthologs for rat genes have been annotated and curated by the Mouse Genome Informatics (MGI) group at The Jackson Laboratories and described at the web-site at URL www.informaticsjax.org. The ortholog data has been used to create high density comparative maps between genes found in rat and human species, as described by Kwitek et al., Genome Research Vol. 11(11): 1935-1943 (2001), which is hereby incorporated by reference herein.

### III. General Methods of the Invention

The present invention provides a method to derive multiple non-overlapping gene signatures for non-genotoxic hepatocarcinogenicity. These non-overlapping signatures use different genes and thus each may be used independently in a predictive assay to confirm that an individual will suffer non-genotoxic hepatocarcinogenicity. Furthermore, this method for identifying non-overlapping gene signatures also provides the list of all genes “necessary” to create a signature that performs above a certain minimal threshold level for a specific predicting whether an organism, cell or tissue culture will exhibit the biological activity, response, or endpoint indicating non-genotoxic hepatocarcinogenicity. This necessary set of genes also may be used to derive additional signatures with varying numbers of genes and levels of performance for particular applications (e.g., diagnostic assays and devices).

Classifiers comprising genes as variables and accompanying weighting factors may be used to classify large datasets compiled from DNA microarray experiments. Of particular preference are sparse linear classifiers. Sparse as used here means that the vast majority of the genes measured in the expression experiment have zero weight in the final linear classifier. Sparsity ensures that the sufficient and necessary gene lists produced by the methodology described herein are as short as possible. These short weighted gene lists (i.e., a gene signature) are capable of assigning an unknown compound treatment to one of two classes.

The sparsity and linearity of the classifiers are important features. The linearity of the classifier facilitates the interpretation of the signature—the contribution of each gene to the classifier corresponds to the product of its weight and the value (i.e., log₁₀ ratio) from the microarray experiment. The property of sparsity ensures that the classifier uses only a few genes, which also helps in the interpretation. More importantly, the sparsity of the classifier may be reduced to a practical diagnostic apparatus or device comprising a relatively small set of reagents representing genes.

A. Gene Expression Related Datasets

a. Various Useful Data Types

The present invention may be used with a wide range of gene expression related data types to generate necessary and sufficient sets of genes useful for non-genotoxic hepatocarcinogenicity signatures. In a preferred embodiment, the present invention utilizes data generated by high-throughput biological assays such as DNA microarray experiments, or proteomic assays. The datasets are not limited to gene expression related data but also may include any sort of molecular characterization information including, e.g., spectroscopic data (e.g., UV-Vis, NMR, IR, mass spectrometry, etc.), structural data (e.g., three-dimensional coordinates) and functional data (e.g., activity assays, binding assays). The gene sets and signatures produced by using the present invention may be applied in a multitude of analytical contexts, including the development and manufacture of detection devices (i.e., diagnostics).

b. Construction of a Gene Expression Dataset

The present invention may be used to identify necessary and sufficient sets of responsive genes within a gene expression dataset that are useful for predicting non-genotoxic hepatocarcinogenicity. For example, the data may correspond to treatments of cells, tissues, or whole organisms (e.g., cells, worms, frogs, mice, rats, primates, or humans etc.) with chemical compounds at varying dosages and times followed by gene expression profiling of the organism's transcriptome (e.g., measuring mRNA levels) or proteome (e.g., measuring protein levels). In the case of multicellular organisms (e.g., mammals) the expression profiling may be carried out on various tissues of interest (e.g., liver, kidney, marrow, spleen, heart, brain, intestine). Typically, valid sufficient classifiers or signatures may be generated that answer questions relevant to classifying treatments in a single tissue type. The present specification describes examples of necessary and sufficient gene signatures useful for classifying chemogenomic data in liver tissue. The methods of the present invention may also be used however, to generate signatures in any tissue type. In some embodiments, classifiers or signatures may be useful in more than one tissue type. Indeed, a large chemogenomic dataset, like that exemplified in the present invention may reveal gene signatures in one tissue type (e.g., liver) that also classify pathologies in other tissues (e.g., intestine).

A gene signature can be generated using a data set derived from chemogenomic experiments on rats and an experimental device useful for detecting a biological responses can be created using the corresponding genes of rat or orthologs of rat genes found in another organism (e.g., human). The present invention can also be used to assay experimental data generated from a variety of experimental sources, including treatments of cells, tissue cultures, or whole in vivo treated organisms.

The present invention describes methods of analysis comprising comparing the experimental expression data to a database of expression data and then querying by means of a computer interface for corresponding patterns associated with defined gene signatures.

In addition to the expression profile data, the present invention may be useful with chemogenomic datasets including additional data types such as data from classic biochemistry assays carried out on the organisms and/or tissues of interest. Other data included in a large multivariate dataset may include histopathology, pharmacology assays, and structural data for the chemical compounds of interest.

One example of a chemogenomic multivariate dataset particularly useful with the present invention is a dataset based on DNA array expression profiling data as described in U.S. patent publication 2002/0174096 A1, published Nov. 21, 2002, and U.S. patent publication 2005/0060102 A1, published Mar. 17, 2005, each of which is hereby incorporated by reference for all purposes. Microarrays are well known in the art and consist of a substrate to which probes that correspond in sequence to genes or gene products (e.g., cDNAs, mRNAs, cRNAs, polypeptides, and fragments thereof), can be specifically hybridized or bound at a known position. The microarray is an array (i.e., a matrix) in which each position represents a discrete binding site for a gene or gene product (e.g., a DNA or protein), and in which binding sites are present for many or all of the genes in an organism's genome.

As disclosed above, a treatment may include but is not limited to the exposure of a biological sample or organism (e.g., a rat) to a drug candidate (or other chemical compound), the introduction of an exogenous gene into a biological sample, the deletion of a gene from the biological sample, or changes in the culture conditions of the biological sample. Responsive to a treatment, a gene corresponding to a microarray site may, to varying degrees, be (a) up-regulated, in which more mRNA corresponding to that gene may be present, (b) down-regulated, in which less mRNA corresponding to that gene may be present, or (c) unchanged. The amount of up-regulation or down-regulation for a particular matrix location is made capable of machine measurement using known methods (e.g., fluorescence intensity measurement). For example, a two-color fluorescence detection scheme is disclosed in U.S. Pat. Nos. 5,474,796 and 5,807,522, both of which are hereby incorporated by reference herein. Single color schemes are also well known in the art, wherein the amount of up- or down-regulation is determined in silico by calculating the ratio of the intensities from the test array divided by those from a control.

After treatment and appropriate processing of the microarray, the photon emissions are scanned into numerical form, and an image of the entire microarray is stored in the form of an image representation such as a color JPEG or TIFF format. The presence and degree of up-regulation or down-regulation of the gene at each microarray site represents, for the perturbation imposed on that site, the relevant output data for that experimental run or scan.

The methods for reducing datasets disclosed herein are broadly applicable to other gene and protein expression data. For example, in addition to microarray data, biological response data including gene expression level data generated from serial analysis of gene expression (SAGE, supra) (Velculescu et al., 1995, Science, 270:484) and related technologies are within the scope of the multivariate data suitable for analysis according to the method of the invention. Other methods of generating biological response signals suitable for the preferred embodiments include, but are not limited to: traditional Northern and Southern blot analysis; antibody studies; chemiluminescence studies based on reporter genes such as luciferase or green fluorescent protein; Lynx; READS (Gene Logic, Inc., Gaithersburg, Md.); and methods similar to those disclosed in U.S. Pat. No. 5,569,588 to Ashby et. al., “Methods for drug screening,” which are hereby incorporated by reference into the present disclosure.

In another preferred embodiment, the large multivariate dataset may include genotyping (e.g., single-nucleotide polymorphism) data. The present invention may be used to generate necessary and sufficient sets of variables capable of classifying genotype information. These signatures would include specific high-impact SNPs that could be used in a genetic diagnostic or pharmacogenomic assay.

The method of generating classifiers from a multivariate dataset according to the present invention may be aided by the use of relational database systems (e.g., in a computing system) for storing and retrieving large amounts of data. The advent of high-speed wide area networks and the internet, together with the client/server based model of relational database management systems, is particularly well-suited for meaningfully analyzing large amounts of multivariate data given the appropriate hardware and software computing tools. Computerized analysis tools are particularly useful in experimental environments involving biological response signals (e.g., absolute or relative gene expression levels). Generally, multivariate data may be obtained and/or gathered using typical biological response signals. Responses to biological or environmental stimuli may be measured and analyzed in a large-scale fashion through computer-based scanning of the machine-readable signals, e.g., photons or electrical signals, into numerical matrices, and through the storage of the numerical data into relational databases. For example a large chemogenomic dataset may be constructed as described in U.S. patent publication 2005/0060102, published Mar. 17, 2005, which is hereby incorporated by reference for all purposes.

B. Generating Valid Gene Signatures from a Chemogenomic Dataset

a. Mining a Large Chemogenomic Dataset

Generally classifiers or signatures are generated (i.e., mined) from a large multivariate dataset by first labeling the full dataset according to known classifications and then applying an algorithm to the full dataset that produces a linear classifier for each particular classification question. Each signature so generated is then cross-validated using a standard split sample procedure.

The initial questions used to classify (i.e., the classification questions) a large multivariate dataset may be of any type susceptible to yielding a yes or no answer. The general form of such questions is: “Is the unknown a member of the class or does it belong with everything else outside the class?” For example, in the area of chemogenomic datasets, classification questions may include “mode-of-action” questions such as “All treatments with drugs belonging to a particular structural class versus the rest of the treatments” or pathology questions such as “All treatments resulting in a measurable pathology versus all other treatments.” In the specific case of chemogenomic datasets based on gene expression, it is preferred that the classification questions are further categorized based on the tissue source of the gene expression data. Similarly, it may be helpful to subdivide other types of large data sets so that specific classification questions are limited to particular subsets of data (e.g., data obtained at a certain time or dose of test compound). Typically, the significance of subdividing data within large datasets become apparent upon initial attempts to classify the complete dataset. A principal component analysis of the complete data set may be used to identify the subdivisions in a large dataset (see e.g., US 2003/0180808 A1, published Sep. 25, 2003, which is hereby incorporated by reference herein.) Methods of using classifiers to identify information rich genes in large chemogenomic datasets is also described in U.S. Ser. No. 11/114,998, filed Apr. 25, 2005, which is hereby incorporated by reference herein for all purposes.

Labels are assigned to each individual (e.g., each compound treatment) in the dataset according to a rigorous rule-based system. The +1 label indicates that a treatment falls in the class of interest, while a −1 label indicates that the variable is outside the class. Thus, with respect to the 64 compound treatments shown in Table 2 (see Example 2 below) used in generating an non-genotoxic hepatocarcinogenicity signature, the “in-class” treatments were labeled +1, whereas the “not in-class” were labeled −1. Information used in assigning labels to the various individuals to classify may include annotations from the literature related to the dataset (e.g., known information regarding the compounds used in the treatment), or experimental measurements on the exact same animals (e.g., results of clinical chemistry or histopathology assays performed on the same animal). A more detailed description of the general method for using classification questions to mine a chemogenomic dataset for signatures is described in U.S. Ser. No. 11/149,612, filed Jun. 10, 2005, and PCT/US2005/020695, filed Jun. 10, 2005, each of which is hereby incorporated in its entirety by reference herein.

b. Algorithms for Generating Valid Gene Signatures

Dataset classification may be carried out manually, that is by evaluating the dataset by eye and classifying the data accordingly. However, because the dataset may involve tens of thousands (or more) individual variables, more typically, querying the full dataset with a classification question is carried out in a computer employing any of the well-known data classification algorithms.

In preferred embodiments, algorithms are used to query the full dataset that generate linear classifiers. In particularly preferred embodiments the algorithm is selected from the group consisting of: SPLP, SPLR and SPMPM. These algorithms are based respectively on Linear Programming (LP), Logistic Regression (LR) and Minimax Probability Machine (MPM). They have been described in detail elsewhere (See e.g., El Ghaoui et al., op. cit; Brown, M. P., W. N. Grundy, D. Lin, N. Cristianini, C. W. Sugnet, T. S. Furey, M. Ares, Jr., and D. Haussler, “Knowledge-based analysis of microarray gene expression data by using support vector machines,” Proc Natl Acad Sci USA 97: 262-267 (2000)).

Generally, the sparse classification methods SPLP, SPLR, SPMPM are linear classification algorithms in that they determine the optimal hyperplane separating a positive and a negative class. This hyperplane, H can be characterized by a vectorial parameter, w (the weight vector) and a scalar parameter, b (the bias): H={x|w^{T}x+b=0}.

For all proposed algorithms, determining the optimal hyperplane reduces to optimizing the error on the provided training data points, computed according to some loss function (e.g., the “Hinge loss;” the “LR loss;” or the “MPM loss”) augmented with a 1-norm regularization component, w. Regularization helps to provide a sparse, short signature. Moreover, this 1-norm penalty on the signature will be weighted by the average standard error per gene. That is, genes that have been measured with more uncertainty will be less likely to get a high weight in the signature. Consequently, the proposed algorithms lead to sparse signatures, and take into account the average standard error information.

Mathematically, the algorithms can be described by the cost functions (shown below for SPLP, SPLR and SPMPM) that they actually minimize to determine the parameters w and b.

$${min}_{w , b} {\sum }_{i} {e}_{i} + ρ {\sum }_{i} {σ}_{i} \vert {w}_{i} \vert s . t . {y}_{i} ( {w}^{T} {x}_{i} + b ) \geq 1 - {e}_{i} \\ {e}_{i} \geq 0 , \\ i = 1 , … , N \qquad SPLP$$

The first term minimizes the training set error, while the second term is the 1-norm penalty on the signature w, weighted by the average standard error information per gene given by sigma. The training set error is computed according to the so-called Hinge loss, as defined in the constraints. This loss function penalizes every data point that is closer than “1” to the separating hyperplane H, or is on the wrong side of H. Notice how the hyperparameter rho allows trade-off between training set error and sparsity of the signature w.

$${min}_{w , b} {\sum }_{i} log ( 1 + exp ( - {y}_{i} ( {w}^{T} {x}_{i} + b ) ) ) + ρ {\sum }_{i} {σ}_{i} \vert {w}_{i} \vert \qquad SPLR$$

The first term expresses the negative log likelihood of the data (a smaller value indicating a better fit of the data), as usual in logistic regression, and the second term will give rise to a short signature, with rho determining the trade-off between both.

$${min}_{w} \sqrt{{w}^{T} {\hat{Γ}}_{+} w} + \sqrt{{w}^{T} {\hat{Γ}}_{-} w} + ρ {\sum }_{i} {σ}_{i} \vert {w}_{i} \vert \\ s . t . {w}^{T} ( {\hat{x}}_{+} - {\hat{x}}_{-} ) = 1 \qquad SPMPM$$

Here, the first two terms, together with the constraint are related to the misclassification error, while the third term will induce sparsity, as before. The symbols with a hat are empirical estimates of the covariances and means of the positive and the negative class. Given those estimates, the misclassification error is controlled by determining w and b such that even for the worst-case distributions for the positive and negative class (which we do not exactly know here) with those means and covariances, the classifier will still perform well. More details on how this exactly relates to the previous cost function can be found in e.g., El Ghaoui, L., G. R. G. Lanckriet, and G. Natsoulis, 2003, “Robust classifiers with interval data” Report # UCB/CSD-03-1279. Computer Science Division (EECS), University of California, Berkeley, Calif., and PCT publication WO 2005/017807 A2, published Feb. 24, 2005, each of which is hereby incorporated by reference herein.

As mentioned above, classification algorithms capable of producing linear classifiers are preferred for use with the present invention. In the context of chemogenomic datasets, linear classifiers may be used to generate one or more valid signatures capable of answering a classification question comprising a series of genes and associated weighting factors. Linear classification algorithms are particularly useful with DNA array or proteomic datasets because they provide simplified signatures useful for answering a wide variety of questions related to biological function and pharmacological/toxicological effects associated with genes or proteins. These signatures are particularly useful because they are easily incorporated into wide variety of DNA- or protein-based diagnostic assays (e.g., DNA microarrays).

However, some classes of non-linear classifiers, so called kernel methods, may also be used to develop short gene lists, weights and algorithms that may be used in diagnostic device development; while the preferred embodiment described here uses linear classification methods, it specifically contemplates that non-linear methods may also be suitable.

Classifications may also be carried using principle component analysis and/or discrimination metric algorithms well-known in the art (see e.g., US 2003/0180808 A1, published Sep. 25, 2003, which is hereby incorporated by reference herein).

Additional statistical techniques, or algorithms, are known in the art for generating classifiers. Some algorithms produce linear classifiers, which are convenient in many diagnostic applications because they may be represented as a weighted list of variables. In other cases non-linear classifier functions of the initial variables may be used. Other types of classifiers include decision trees and neural networks. Neural networks are universal approximators (Hornik, K., M. Stinchcombe, and H. White. 1989. “Multilayer feedforward networks are universal approximators,” Neural Networks 2: 359-366); they can approximate any measurable function arbitrarily well, and they can readily be used to model classification functions as well. They perform well on several biological problems, e.g., protein structure prediction, protein classification, and cancer classification using gene expression data (see, e.g., Bishop, C. M. 1996. Neural Networks for Pattern Recognition. Oxford University Press; Khan, J., J. S. Wei, M. Ringner, L. H. Saal, M. Ladanyi, F. Westermann, F. Berthold, M. Schwab, C. R. Antonescu, C. Peterson, and P. S. Meltzer. 2001. Classification and diagnostic prediction of cancers using gene expression profiling and artificial neural networks. Nat Med 7: 673-679; Wu, C. H., M. Berry, S. Shivakumar, and J. McLarty. 1995. Neural networks for full-scale protein sequence classification: sequence encoding with singular value decomposition. Machine Learning 21: 177-193).

c. Cross-Validation of Gene Signatures

Cross-validation of a gene signature's performance is an important step for determining whether the signature is sufficient. Cross-validation may be carried out by first randomly splitting the full dataset (e.g., a 60/40 split). A training signature is derived from the training set composed of 60% of the samples and used to classify both the training set and the remaining 40% of the data, referred to herein as the test set. In addition, a complete signature is derived using all the data. The performance of these signatures can be measured in terms of log odds ratio (LOR) or the error rate (ER) defined as:

LOR=ln (((TP+0.5)*(TN+0.5))/((FP+0.5)*(FN+0.5)))

and

ER=(FP+FN)/N;

where TP, TN, FP, FN, and N are true positives, true negatives, false positives, false negatives, and total number of samples to classify, respectively, summed across all the cross validation trials. The performance measures are used to characterize the complete signature, the average of the training or the average of the test signatures.

The algorithms described above are capable of generating a plurality of gene signatures with varying degrees of performance for the classification task. In order to identify that signatures that are to be considered “valid,” a threshold performance is selected for the particular classification question. In one preferred embodiment, the classifier threshold performance is set as log odds ratio greater than or equal to 4.00 (i.e., LOR≧4.00). However, higher or lower thresholds may be used depending on the particular dataset and the desired properties of the signatures that are obtained. Of course many queries of a chemogenomic dataset with a classification question will not generate a valid gene signature.

Two or more valid gene signatures may be generated that are redundant or synonymous for a variety of reasons. Different classification questions (i.e., class definitions) may result in identical classes and therefore identical signatures. For instance, the following two class definitions define the exact same treatments in the database: (1) all treatments with molecules structurally related to statins; and (2) all treatments with molecules having an IC₅₀<1 μM for inhibition of the enzyme HMG CoA reductase.

In addition, when a large dataset is queried with the same classification question using different algorithms (or even the same algorithm under slightly different conditions) different, valid signatures may be obtained. These different signatures may or may not comprise overlapping sets of variables; however, they each can accurately identify members of the class of interest.

For example, as illustrated in Table 1, two equally performing gene signatures (LOR=˜7.0) for the fibrate class of compounds may be generated by querying a chemogenomic dataset with two different algorithms: SPLP and SPLR. Genes are designated by their accession number and a brief description. The weights associated with each gene are also indicated. Each signature was trained on the exact same 60% of the multivariate dataset and then cross validated on the exact same remaining 40% of the dataset. Both signatures were shown to exhibit the exact same level of performance as classifiers: two errors on the cross validation data set. The SPLP derived signature consists of 20 genes. The SPLR derived signature consists of eight genes. Only three of the genes from the SPLP signature are present in the eight gene SPLR signature.

TABLE 1
Two Gene Signatures for the Fibrate Class of Drugs

| | Accession | Weight | Unigene name |
|---|---|---|---|
| SPLP | K03249 | 1.1572 | enoyl-Co A, hydratase/3-hydroxyacyl Co A dehydrogenase |
| | AW916833 | 1.0876 | hypothetical protein RMT-7 |
| | BF387347 | 0.4769 | ESTs |
| | BF282712 | 0.4634 | ESTs |
| | AF034577 | 0.3684 | pyruvate dehydrogenate kinase 4 |
| | NM_019292 | 0.3107 | carbonic anhydrase 3 |
| | AI179988 | 0.2735 | ectodermal-neural cortex (with BTB-like domain) |
| | AI715955 | 0.211 | Stac protein (SRC homology 3 and cysteine-rich domain protein) |
| | BE110695 | 0.2026 | activating transcription factor 1 |
| | J03752 | 0.0953 | microsomal glutathione S-transferase 1 |
| | D86580 | 0.0731 | nuclear receptor subfamily 0, group B, member 2 |
| | BF550426 | 0.0391 | KDEL (Lys-Asp-Glu-Leu) endoplasmic reticulum protein retention receptor 2 |
| | AA818999 | 0.0296 | muscleblind-like 2 |
| | NM_019125 | 0.0167 | probasin |
| | AF150082 | −0.0141 | translocase of inner mitochondrial membrane 8 (yeast) homolog A |
| | BE118425 | −0.0781 | Arsenical pump-driving ATPase |
| | NM_017136 | −0.126 | squalene epoxidase |
| | AI171367 | −0.3222 | HSPC154 protein |
| | NM_019369 | −0.637 | inter alpha-trypsin inhibitor, heavy chain 4 |
| | AI137259 | −0.7962 | ESTs |
| SPLR | NM_017340 | 5.3688 | acyl-coA oxidase |
| | BF282712 | 4.1052 | ESTs |
| | NM_012489 | 3.8462 | acetyl-Co A acyltransferase 1 (peroxisomal 3-oxoacyl-Co A thiolase) |
| | BF387347 | 1.767 | ESTs |
| | K03249 | 1.7524 | enoyl-Co A, hydratase/3-hydroxyacyl Co A dehydrogenase |
| | NM_016986 | 0.0622 | acetyl-co A dehydrogenase, medium chain |
| | AB026291 | −0.7456 | acetoacetyl-CoA synthetase |
| | AI454943 | −1.6738 | likely ortholog of mouse porcupine homolog |

It is interesting to note that only three genes are common between these two signatures, (K03249, BF282712, and BF387347) and even those are associated with different weights. While many of the genes may be different, some commonalities may nevertheless be discerned. For example, one of the negatively weighted genes in the SPLP derived signature is NM_{—}017136 encoding squalene epoxidase, a well-known cholesterol biosynthesis gene. Squalene epoxidase is not present in the SPLR derived signature but aceto-acetyl CoA synthetase, another cholesterol biosynthesis gene is present and is also negatively weighted.

Additional variant signatures may be produced for the same classification task. For example, the average signature length (number of genes) produced by SPLP and SPLR, as well as the other algorithms (e.g., “adjusted” SPLP or A-SPLP), may be varied by use of the parameter ρ (see e.g., El Ghaoui, L., G. R. G. Lanckriet, and G. Natsoulis, 2003, “Robust classifiers with interval data” Report # UCB/CSD-03-1279. Computer Science Division (EECS), University of California, Berkeley, Calif.; PCT publication WO 2005/017807 A2, published Feb. 24, 2005, and U.S. Ser. No. 11/332,718, filed Jan. 12, 2006, each of which is hereby incorporated by reference herein). Varying ρ can produce signatures of different length with comparable test performance (Natsoulis et al., “Classification of a large microarray data set: Algorithm comparison and analysis of drug signatures,” Gen. Res. 15:724-736 (2005)). Those signatures are obviously different and often have no common genes between them (i.e., they do not overlap in terms of genes used).

C. “Stripping” Signatures from a Dataset to Generate the “Necessary” Set

Each individual classifier or signature is capable of classifying a dataset into one of two categories or classes defined by the classification question. Typically, an individual signature with the highest test log odds ratio will be considered as the best classifier for a given task. However, often the second, third (or lower) ranking signatures, in terms of performance, may be useful for confirming the classification of compound treatment, especially where the unknown compound yields a borderline answer based on the best classifier. Furthermore, the additional signatures may identify alternative sources of informational rich data associated with the specific classification question. For example, a slightly lower ranking gene signature from a chemogenomic dataset may include those genes associated with a secondary metabolic pathway affected by the compound treatment. Consequently, for purposes of fully characterizing a class and answering difficult classification questions, it is useful to define the entire set of variables that may be used to produce the plurality of different classifiers capable of answering a given classification question. This set of variables is referred to herein as a “necessary set.” Conversely, the remaining variables from the full dataset are those that collectively cannot be used to produce a valid classifier, and therefore are referred to herein as the “depleted set.”

The general method for identifying a necessary set of variables useful for a classification question involved what is referred to herein as a classifier “stripping” algorithm. The stripping algorithm comprises the following steps: (1) querying the full dataset with a classification question so as to generate a first linear classifier capable of performing with a log odds ratio greater than or equal to 4.0 comprising a first set of variables; (2) removing the variables of the first linear classifier from the full dataset thereby generating a partially depleted dataset; (3) re-querying the partially depleted dataset with the same classification question so as to generate a second linear classifier and cross-validating this second classifier to determine whether it performs with a log odds ratio greater than or equal to 4. If it does not, the process stops and the dataset is fully depleted for variables capable of generating a classifier with an average log odds ratio greater than or equal to 4.0. If the second classifier is validated as performing with a log odds ratio greater than or equal to 4.0, then its variables are stripped from the full dataset and the partially depleted set if re-queried with the classification question. These cycles of stripping and re-querying are repeated until the performance of any remaining set of variables drops below an arbitrarily set LOR. The threshold at which the iterative process is stopped may be arbitrarily adjusted by the user depending on the desired outcome. For example, a user may choose a threshold of LOR=0. This is the value expected by chance alone. Consequently, after repeated stripping until LOR=0 there is no classification information remaining in the depleted set. Of course, selecting a lower value for the threshold will result in a larger necessary set.

Although a preferred cut-off for stripping classifiers is LOR=4.0, this threshold is arbitrary. Other embodiments within the scope of the invention may utilize higher or lower stripping cutoffs e.g., depending on the size or type of dataset, or the classification question being asked. In addition other metrics could be used to assess the performance (e.g., specificity, sensitivity, and others). Also the stripping algorithm removes all variables from a signature if it meets the cutoff. Other procedures may be used within the scope of the invention wherein only the highest weighted or ranking variables are stripped. Such an approach based on variable impact would likely result in a classifier “surviving” more cycles and defining a smaller necessary set.

Other procedures may be used within the scope of the invention wherein only the highest weighted or ranking variables are stripped. Such an approach based on variable impact would likely result in a classifier “surviving” more cycles and defining a smaller necessary set.

In another alternative approach, the genes from signatures may be stripped from the dataset until it is unable to generate a signature capable of classifying the “true label set” with an LOR that is statistically different from its classification of the “random label set.” The “true label set” refers to a training set of compound treatment data that is correctly labeled (e.g., +1 class, −1 class) for the particular classification question. The “random label set” refers to the same set of compound treatment data where the class labels have been randomly assigned. Attempts to use a signature to classify a random label set will result in an average LOR of approximately zero and some standard deviation (SD). These values may be compared to the average LOR and SD for the classifying the true label set, where the SD is calculated based on LOR results across the 20 or 40 splits. The difference in classifying true and random label sets with valid signatures should be significantly greater than random. In such an alternative approach, the selected performance threshold for a signature is a p-value rather than a LOR cutoff.

The resulting fully-depleted set of variables that remains after a classifier is fully stripped from the full dataset cannot generate a classifier for the specific classification question (with the desired level of performance). Consequently, the set of all of the variables in the classifiers that were stripped from the full set are defined as “necessary” for generating a valid classifier.

The stripping method utilizes a classification algorithm at its core. The examples presented here use SPLP or A-SPLP for this task. Other algorithms, provided that they are sparse with respect to genes could be employed. SPLR and SPMPM are two alternatives for this functionality (see e.g., El Ghaoui, L., G. R. G. Lanckriet, and G. Natsoulis, 2003, “Robust classifiers with interval data” Report # UCB/CSD-03-1279. Computer Science Division (EECS), University of California, Berkeley, Calif., PCT publication WO 2005/017807 A2, published Feb. 24, 2005, and U.S. Ser. No. 11/332,718, filed Jan. 12, 2006, each of which is hereby incorporated by reference herein).

In one embodiment, the stripping algorithm may be used on a chemogenomics dataset comprising DNA microarray data. The resulting necessary set of genes comprises a subset of highly informative genes for a particular classification question. Consequently, these genes may be incorporated in diagnostic devices (e.g., polynucleotide arrays) where that particular classification (e.g., non-genotoxic hepatocarcinogenicity) is of interest. In other exemplary embodiments, the stripping method may be used with datasets from proteomic experiments.

D. Mining the Non-Genotoxic Hepatocarcinogenicity Necessary Set for Signatures

Besides identifying the “necessary” set of genes for a particular signature (i.e., classifier), another important use of the stripping algorithm is the identification of multiple, non-overlapping sufficient sets of genes useful for answering a particular classification question. These non-overlapping sufficient sets are a direct product of the above-described general method of stripping valid classifiers. Because the method iteratively eliminates the genes of the prior derived valid classifier, each subsequent iteration of the method results in a valid classifier comprising all new genes. Typically, the earlier stripped non-overlapping gene signature yields higher performance with fewer genes. In other words, the earliest identified sufficient set usually comprises the highest impact, most information-rich genes with respect to the particular classification question. The valid classifiers that appear during later iterations of the stripping algorithm typically contain a larger number of genes. However, these later appearing classifiers may provide valuable information regarding normally unrecognized relationships between genes in the dataset. For example, in the case of non-overlapping gene signatures identified by stripping in a chemogenomics dataset, the later appearing signatures may include families of genes not previously recognized as involved in the particular metabolic pathway that is being affected by a particular compound treatment. Thus, chemogenomic data sets may be stripped to identify underlying signatures that contain novel genes previously not associated with a particular non-genotoxic hepatocarcinogenicity. This type of functional analysis via the stripping procedure may identify new metabolic targets associated with a compound treatment.

The necessary set high impact genes generated by the stripping method itself represents a subset of genes that may be mined for further signatures. Hence, the complete set of genes in a necessary set for predicting non-genotoxic hepatocarcinogenicity may used to randomly generate random subsets of genes of varying size that are capable of generating additional predictive signatures. One preferred method of selecting such subsets is based on percentage of total impact. Thus, subsets of genes are selected whose summed impact factors are a selected percentage of the total impact (i.e., the sum of the impacts of all genes in the necessary set). These percentage impact subsets may be used to generate new signatures for predicting non-genotoxic hepatocarcinogenicity. For example, a random subset of genes selected from the necessary set with 4% of the total impact may be used with one of the linear programming algorithms (e.g., A-SPLP) to generate a new linear classifier of 8 genes, weighting factors and a bias term that may be used as a signature for non-genotoxic hepatocarcinogenicity. Thus, the necessary set for a particular classification represents a greatly reduced dataset that can generate new signatures with varying properties such as shorter (or longer) gene lengths and higher (or lower) LOR performance values.

E. Functional Characterization of the Non-Genotoxic Hepatocarcinogenicity Necessary Set

The above-described stripping method produces a necessary set of genes for answering the non-genotoxic hepatocarcinogenicity classification question. This necessary set of genes also may be characterized functionally based on the ability of the information rich genes in the set to “revive” the ability of a fully “depleted” set of genes to generate valid non-genotoxic hepatocarcinogenicity signatures. Thus, the necessary set for the non-genotoxic hepatocarcinogenicity classification question corresponds to that set of genes from which any random selection when added to a depleted set (i.e., depleted for non-genotoxic hepatocarcinogenicity classification question) restores the ability of that set to produce non-genotoxic hepatocarcinogenicity signatures with an average LOR (avg LOR) above a threshold level. The general method for functionally characterizing a necessary set in terms of its ability to revive its depleted set is described in U.S. Ser. No. 11/149,612, filed Jun. 10, 2005, and PCT/US2005/020695, filed Jun. 10, 2005, each of which is hereby incorporated in its entirety by reference herein.

Preferably, the threshold performance used is an average logodds ratio (“avg LOR”) greater than or equal to 4.00. Other values for performance, however, may be set. For example, avg LOR may vary from about 1.0 to as high as 8.0. In preferred embodiments, the avg LOR threshold may be 3.0 to as high as 7.0 including all integer and half-integer values in that range. The necessary set may then be defined in terms of percentage of randomly selected genes from the necessary set that restore the performance of a depleted set above a certain threshold. Typically, the avg LOR of the depleted set is ˜1.20, although as mentioned above, datasets may be depleted more or less depending on the threshold set, and depleted sets with avg LOR as low as 0.0 may be used. Generally, the depleted set will exhibit an avg LOR between about 0.5 and 1.5.

The third parameter establishing the functional characteristics of the non-genotoxic hepatocarcinogenicity necessary set of genes for answering the non-genotoxic hepatocarcinogenicity classification question is the percentage of randomly selected genes from that set that result in reviving the threshold performance of the depleted set. For example, where the threshold avg LOR is at least 4.00 and the depleted set performs with an avg LOR of ˜1.20, typically 16-36% of randomly selected genes from the necessary set are required to restore the average performance of the depleted set to the threshold value. In preferred embodiments, the random supplementation may be achieved using 16, 18, 20, 22, 24, 26, 28, 30, 32, 34 or 36% of the necessary set.

Alternatively, as described above, the necessary set may be characterized based on its ability to randomly generate signatures capable of classifying a true label set with an average performance above those signatures ability to classify a random label set. In preferred embodiments, signatures generated from a random selection of at least 10% of the genes in the necessary set may perform at least 1 standard deviation, and preferably at least 2 standard deviations, better for classifying the true versus the random label set. In other embodiments, the random selection may be of at least 15%, 20%, 25%, 30%, 40%, 50%, and even higher percentages of genes from the set.

F. Using Signatures and the Necessary Set to Generate Diagnostic Assays and Devices for Predicting Non-Genotoxic Hepatocarcinogenicity

A diagnostic usually consists in performing one or more assays and in assigning a sample to one or more categories based on the results of the assay(s). Desirable attributes of a diagnostic assays include high sensitivity and specificity measured in terms of low false negative and false positive rates and overall accuracy. Because diagnostic assays are often used to assign large number of samples to given categories, the issues of cost per assay and throughput (number of assays per unit time or per worker hour) are of paramount importance.

Typically the development of a diagnostic assay involves the following steps: (1) define the end point to diagnose, e.g., cholestasis, a pathology of the liver (2) identify one or more markers whose alteration correlates with the end point, e.g., elevation of bilirubin in the bloodstream as an indication of cholestasis; and (3) develop a specific, accurate, high-throughput and cost-effective assay for that marker. In order to increase throughput and decrease costs several diagnostics are often combined in a panel of assays, especially when the detection methodologies are compatible. For example several ELISA-based assays, each using different antibodies to ascertain different end points may be combined in a single panel and commercialized as a single kit. Even in this case, however, each of the ELISA-based assays had to be developed individually often requiring the generation of specific reagents.

The present invention provides signatures and methods for identifying additional signatures comprising as few as 4 genes that are useful for determining a therapeutic or toxicological end-point for non-genotoxic hepatocarcinogenicity. These signatures (and the genes from which they are composed) may also be used in the design of improved diagnostic devices that answer the same questions as a large microarray but using a much smaller fraction of data. Generally, the reduction of information in a large chemogenomic dataset to a simple signature enables much simpler devices compatible with low cost high throughput multi-analyte measurement.

As described herein, a large chemogenomic dataset may be mined for a plurality of informative genes useful for answering classification questions. The size of the classifiers or signatures so generated may be varied according to experimental needs. In addition, multiple non-overlapping classifiers may be generated where independent experimental measures are required to confirm a classification. Generally, the sufficient classifiers result in a substantial reduction of data that needs to be measured to classify a sample. Consequently, the signatures and methods of the present invention provide the ability to produce cheaper, higher throughput, diagnostic measurement methods or strategies. In particular, the invention provides diagnostic reagent sets useful in diagnostic assays and the associated diagnostic devices and kits. As used herein, diagnostic assays includes assays that may be used for patient prognosis and therapeutic monitoring.

Diagnostic reagent sets may include reagents representing the subset of genes found in the necessary set consisting of less than 50%, 40%, 30%, 20%, 10%, or even less than 5% of the total genes. In one preferred embodiment, the diagnostic reagent set is a plurality of polynucleotides or polypeptides representing specific genes in a sufficient or necessary set of the invention. Such biopolymer reagent sets are immediately applicable in any of the diagnostic assay methods (and the associate kits) well known for polynucleotides and polypeptides (e.g., DNA arrays, RT-PCR, immunoassays or other receptor based assays for polypeptides or proteins). For example, by selecting only those genes found in a smaller yet “sufficient” gene signature, a faster, simpler and cheaper DNA array may be fabricated for that signature's specific classification task. Thus, a very simple diagnostic array may be designed that answers 3 or 4 specific classification questions and includes only 60-80 polynucleotides representing the approximately 20 genes in each of the signatures. Of course, depending on the level of accuracy required the LOR threshold for selecting a sufficient gene signature may be varied. A DNA array may be designed with many more genes per signature if the LOR threshold is set at e.g., 7.00 for a given classification question. The present invention includes diagnostic devices based on gene signatures exhibiting levels of performance varying from less than LOR=3.00 up to LOR=10.00 and greater.

In addition, reagent sets for detecting or monitoring treatment for non-genotoxic hepatocarcinogenicity in humans (or other non-rat species) may be prepared using the human orthologs of the genes in the signatures and necessary set derived in accordance with the methods described herein.

The diagnostic reagent sets of the invention may be provided in kits, wherein the kits may or may not comprise additional reagents or components necessary for the particular diagnostic application in which the reagent set is to be employed. Thus, for a polynucleotide array applications, the diagnostic reagent sets may be provided in a kit which further comprises one or more of the additional requisite reagents for amplifying and/or labeling a microarray probe or target (e.g., polymerases, labeled nucleotides, and the like).

A variety of array formats (for either polynucleotides and/or polypeptides) are well-known in the art and may be used with the methods and subsets produced by the present invention. In one preferred embodiment, photolithographic or micromirror methods may be used to spatially direct light-induced chemical modifications of spacer units or functional groups resulting in attachment at specific localized regions on the surface of the substrate. Light-directed methods of controlling reactivity and immobilizing chemical compounds on solid substrates are well-known in the art and described in U.S. Pat. Nos. 4,562,157, 5,143,854, 5,556,961, 5,968,740, and 6,153,744, and PCT publication WO 99/42813, each of which is hereby incorporated by reference herein. Alternatively, a plurality of molecules may be attached to a single substrate by precise deposition of chemical reagents. For example, methods for achieving high spatial resolution in depositing small volumes of a liquid reagent on a solid substrate are disclosed in U.S. Pat. Nos. 5,474,796 and 5,807,522, both of which are hereby incorporated by reference herein. Methods for selecting probes and performing a variety of hybridization-based assays on arrays are well-known in the art and described in e.g., U.S. Pat. Nos. 6,500,938 and 6,635,423, both of which are hereby incorporated by reference herein. In particular, methods for using arrays in a highly reproducible, high-throughput fashion to carry out chemogenomic analyses are described in U.S. Published Patent Appl. 2005/0060102 A1, which is hereby incorporated by reference herein.

It should also be noted that in many cases a single diagnostic device may not satisfy all needs. However, even for an initial exploratory investigation (e.g., classifying drug-treated rats) DNA arrays with sufficient gene sets of varying size (number of genes), each adapted to a specific follow-up technology, can be created. In addition, in the case of drug-treated rats, different arrays may be defined for each tissue.

Alternatively, a single substrate may be produced with several different small arrays of genes in different areas on the surface of the substrate. Each of these different arrays may represent a sufficient set of genes for the same classification question but with a different optimal gene signature for each different tissue. Thus, a single array could be used for particular diagnostic question regardless of the tissue source of the sample (or even if the sample was from a mixture of tissue sources, e.g., in a forensic sample).

In addition, it may be desirable to investigate classification questions of a different nature in the same tissue using several arrays featuring different non-overlapping gene signatures for a particular classification question.

As described above, the methodology described here is not limited to chemogenomic datasets and DNA microarray data. The invention may be applied to other types of datasets to produce necessary and sufficient sets of variables useful for classifiers. For example, proteomics assay techniques, where protein levels are measured or protein interaction techniques such as yeast 2-hybrid or mass spectrometry also result in large, highly multivariate dataset, which could be classified in the same way described here. The result of all the classification tasks could be submitted to the same methods of signature generation and/or classifier stripping in order to define specific sets of proteins useful as signatures for specific classification questions.

In addition, the invention is useful for many traditional lower throughput diagnostic applications. Indeed the invention teaches methods for generating valid, high-performance classifiers consisting of 5% or less of the total variables in a dataset. This data reduction is critical to providing a useful analytical device. For example, a large chemogenomic dataset may be reduced to a signature comprising less than 5% of the genes in the full dataset. Further reductions of these genes may be made by identifying only those genes whose product is a secreted protein. These secreted proteins may be identified based on known annotation information regarding the genes in the subset. Because the secreted proteins are identified in the sufficient set useful as a signature for a particular classification question, they are most useful in protein based diagnostic assays related to that classification. For example, an antibody-based blood serum assay may be produced using the subset of the secreted proteins found in the sufficient signature set. Hence, the present invention may be used to generate improved protein-based diagnostic assays from DNA array information.

The general method of the invention as described above is exemplified below. The following examples are offered as illustrations of specific embodiments and are not intended to limit the inventions disclosed throughout the whole of the specification.

### EXAMPLES

### Example 1

### Construction of a Chemogenomic Reference Database (DrugMatrix™)

This example illustrates the construction of a large multivariate chemogenomic dataset based on DNA microarray analysis of rat tissues from over 580 different in vivo compound treatments. This dataset was used to generate non-genotoxic hepatocarcinogenicity signatures comprising genes and weights which subsequently were used to generate a necessary set of highly responsive genes that may be incorporated into high throughput diagnostic devices as described in Examples 2-7.

The detailed description of the construction of this chemogenomic dataset is described in Examples 1 and 2 of Published U.S. Pat. Appl. No. 2005/0060102 A1, published Mar. 17, 2005, which is hereby incorporated by reference for all purposes. Briefly, in vivo short-term repeat dose rat studies were conducted on over 580 test compounds, including marketed and withdrawn drugs, environmental and industrial toxicants, and standard biochemical reagents. Rats (three per group) were dosed daily at either a low or high dose. The low dose was an efficacious dose estimated from the literature and the high dose was an empirically-determined maximum tolerated dose, defined as the dose that causes a 50% decrease in body weight gain relative to controls during the course of the 5 day range finding study. Animals were necropsied on days 0.25, 1, 3, and 5 or 7. Up to 13 tissues (e.g., liver, kidney, heart, bone marrow, blood, spleen, brain, intestine, glandular and nonglandular stomach, lung, muscle, and gonads) were collected for histopathological evaluation and microarray expression profiling on the Amersham CodeLink™ RU1 platform (also referred to herein as “CodeLink Uniset™ Rat 1 Bioarray”). In addition, a clinical pathology panel consisting of 37 clinical chemistry and hematology parameters was generated from blood samples collected on days 3 and 5.

In order to assure that all of the dataset is of high quality a number of quality metrics and tests are employed. Failure on any test results in rejection of the array and exclusion from the data set. The first tests measure global array parameters: (1) average normalized signal to background, (2) median signal to threshold, (3) fraction of elements with below background signals, and (4) number of empty spots. The second battery of tests examines the array visually for unevenness and agreement of the signals to a tissue specific reference standard formed from a number of historical untreated animal control arrays (correlation coefficient>0.8). Arrays that pass all of these checks are further assessed using principle component analysis versus a dataset containing seven different tissue types; arrays not closely clustering with their appropriate tissue cloud are discarded.

Data collected from the scanner is processed by the Dewarping/Detrending™ normalization technique, which uses a non-linear centralization normalization procedure (see, Zien et al., “Centralization: A new method for the normalization of gene expression data,” in Bioinformatics (2001)) adapted specifically for the CodeLink microarray platform. The procedure utilizes detrending and dewarping algorithms to adjust for non-biological trends and non-linear patterns in signal response, leading to significant improvements in array data quality.

Log₁₀-ratios are computed for each gene as the difference of the averaged logs of the experimental signals from (usually) three drug-treated animals and the averaged logs of the control signals from (usually) 20 mock vehicle-treated animals. To assign a significance level to each gene expression change, the standard error for the measured change between the experiments and controls is computed. An empirical Bayesian estimate of standard deviation for each measurement is used in calculating the standard error, which is a weighted average of the measurement standard deviation for each experimental condition and a global estimate of measurement standard deviation for each gene determined over thousands of arrays (Carlin, and Louis, “Bayes and empirical Bayes methods for data analysis,” Chapman & Hall/CRC, Boca Raton (2000); Gelman, “Bayesian data analysis,” Chapman & Hall/CRC, Boca Raton (1995)). The standard error is used in a t-test to compute a p-value for the significance of each gene expression change. The coefficient of variation (CV) is defined as the ratio of the standard error to the average Log₁₀-ratio, as defined above.

### Example 2

### Preparation of a Chemogenomic Training Dataset for Non-Genotoxic Hepatocarcinogenicity

This example describes methods used to prepare a chemogenomic dataset (i.e., a positive training set) for use deriving a signature for non-genotoxic hepatocarcinogenicity

Overview

To develop an mRNA-based signature to predict non-genotoxic hepatocarcinogens, the literature was curated to annotate compounds in DrugMatrix (Ganter et al., “Development of a large-scale chemogenomics database to improve drug candidate selection and to understand mechanisms of chemical toxicity and action,” J Biotechnol. 119(3):219-44 (2005); and U.S. Pat. Appl. No. 2005/0060102 A1, published Mar. 17, 2005, which is hereby incorporated by reference for all purposes) as being positive or negative for induction of neoplastic lesions in the liver of rats based on two year rodent bioassay results and/or class effect information. Of the 147 compounds annotated, 25 non-genotoxic hepatocarcinogens (NGTC) and 75 non-hepatocarcinogens (NC) were randomly chosen to form a training set (Table 2). The remaining 21 NGTC chemicals and 26 NC chemicals were reserved for independent validation of the signature. Each chemical was evaluated in a short term repeat dose in vivo study in male Sprague-Dawley rats (n=3/group). The livers from treated and control rats were collected after 1, 3 and 5 days of dosing and RNA was extracted for gene expression profiling. Gene expression changes in treated animals were compared to suitably matched controls and represented as average log₁₀ ratios for each treatment group. Since the development of tumors is a long term multi-step process, the 100 training set chemicals were profiled at the latest time point in order to capture gene expression changes that were likely to be reflective of repeat dose treatment effects that contribute to carcinogenicity and not reflective of an initial stimulus following a single dose that may be transient in nature.

The in-class and not in-class compound treatments and are listed in Table 2.

TABLE 2
100 in vivo compound treatments used in the training set.

| | | | | | Time | | |
|---|---|---|---|---|---|---|---|
| Rep | | | | Dose | point | | |
| ID | Set | Class | Compound Name | (mg/kg/d) | (d) | Vehicle | Route |
| 4810 | Training | NGTC | 17- | 2000 | 5 | CMC | ORAL |
| | | | METHYLTESTOSTERONE | | | | GAVAGE |
| 6323 | Training | NGTC | 2,3,7,8- | 0.02 | 5 | CMC | ORAL |
| | | | TETRACHLORODIBENZO- | | | | GAVAGE |
| | | | P-DIOXIN | | | | |
| 5065 | Training | NGTC | ANASTROZOLE | 400 | 5 | CMC | ORAL |
| | | | | | | | GAVAGE |
| 3166 | Training | NGTC | BETA-ESTRADIOL | 150 | 5 | CORN | ORAL |
| | | | | | | OIL | GAVAGE |
| 6060 | Training | NGTC | BUPROPION | 895 | 5 | WATER | ORAL |
| | | | | | | | GAVAGE |
| 2975 | Training | NGTC | CLOFIBRATE | 500 | 7 | CORN | ORAL |
| | | | | | | OIL | GAVAGE |
| 4756 | Training | NGTC | ESTRIOL | 313 | 5 | CMC | ORAL |
| | | | | | | | GAVAGE |
| 2723 | Training | NGTC | ETHANOL | 6000 | 7 | SALINE | ORAL |
| | | | | | | | GAVAGE |
| 2840 | Training | NGTC | ETHINYLESTRADIOL | 1480 | 5 | CORN | ORAL |
| | | | | | | OIL | GAVAGE |
| 5741 | Training | NGTC | ETHISTERONE | 1500 | 5 | CMC | ORAL |
| | | | | | | | GAVAGE |
| 3310 | Training | NGTC | FENOFIBRATE | 215 | 5 | CORN | ORAL |
| | | | | | | OIL | GAVAGE |
| 2878 | Training | NGTC | GEMFIBROZIL | 700 | 7 | CORN | ORAL |
| | | | | | | OIL | GAVAGE |
| 6093 | Training | NGTC | METHAPYRILENE | 100 | 5 | CMC | ORAL |
| | | | | | | | GAVAGE |
| 2819 | Training | NGTC | MIFEPRISTONE | 300 | 5 | CORN | ORAL |
| | | | | | | OIL | GAVAGE |
| 6616 | Training | NGTC | NAFENOPIN | 338 | 5 | CORN | ORAL |
| | | | | | | OIL | GAVAGE |
| 4854 | Training | NGTC | NORETHINDRONE | 125 | 5 | CORN | ORAL |
| | | | ACETATE | | | OIL | GAVAGE |
| 2814 | Training | NGTC | NORETHINDRONE | 375 | 5 | CORN | ORAL |
| | | | | | | OIL | GAVAGE |
| 5445 | Training | NGTC | PENTOBARBITAL | 70 | 5 | WATER | ORAL |
| | | | | | | | GAVAGE |
| 6346 | Training | NGTC | PHENOBARBITAL | 80 | 5 | WATER | ORAL |
| | | | | | | | GAVAGE |
| 6037 | Training | NGTC | PIRINIXIC ACID | 364 | 5 | CMC | ORAL |
| | | | | | | | GAVAGE |
| 5182 | Training | NGTC | PRAVASTATIN | 1200 | 5 | CORN | ORAL |
| | | | | | | OIL | GAVAGE |
| 4945 | Training | NGTC | PREDNISOLONE | 184 | 5 | WATER | ORAL |
| | | | | | | | GAVAGE |
| 3148 | Training | NGTC | PROGESTERONE | 164 | 5 | CORN | ORAL |
| | | | | | | OIL | GAVAGE |
| 4528 | Training | NGTC | SAFROLE | 488 | 5 | CORN | ORAL |
| | | | | | | OIL | GAVAGE |
| 6941 | Training | NGTC | THIOACETAMIDE | 200 | 5 | SALINE | INTRAPERITONEAL |
| 3542 | Training | NC | ACYCLOVIR | 980 | 5 | SALINE | ORAL |
| | | | | | | | GAVAGE |
| 6297 | Training | NC | ALFACALCIDOL | 0.043 | 5 | CMC | ORAL |
| | | | | | | | GAVAGE |
| 6049 | Training | NC | ALLYL ALCOHOL | 32 | 5 | SALINE | INTRAPERITONEAL |
| 3221 | Training | NC | AMLODIPINE | 19 | 5 | CORN | ORAL |
| | | | | | | OIL | GAVAGE |
| 3089 | Training | NC | ASPIRIN | 375 | 5 | CORN | ORAL |
| | | | | | | OIL | GAVAGE |
| 3312 | Training | NC | AZATHIOPRINE | 54 | 5 | WATER | ORAL |
| | | | | | | | GAVAGE |
| 3199 | Training | NC | AZITHROMYCIN | 225 | 5 | WATER | ORAL |
| | | | | | | | GAVAGE |
| 4822 | Training | NC | BENZOIC ACID | 1700 | 5 | CMC | ORAL |
| | | | | | | | GAVAGE |
| 3420 | Training | NC | BISPHENOL A | 610 | 5 | CORN | ORAL |
| | | | | | | OIL | GAVAGE |
| 6101 | Training | NC | CARVEDILOL | 2000 | 5 | CORN | ORAL |
| | | | | | | OIL | GAVAGE |
| 3275 | Training | NC | CATECHOL | 195 | 5 | SALINE | ORAL |
| | | | | | | | GAVAGE |
| 6368 | Training | NC | CELECOXIB | 400 | 5 | CORN | ORAL |
| | | | | | | OIL | GAVAGE |
| 5449 | Training | NC | CERIVASTATIN | 7 | 5 | CORN | ORAL |
| | | | | | | OIL | GAVAGE |
| 4480 | Training | NC | CHOLINE CHLORIDE | 2550 | 5 | WATER | ORAL |
| | | | | | | | GAVAGE |
| 3303 | Training | NC | CIPROFLOXACIN | 450 | 5 | CORN | ORAL |
| | | | | | | OIL | GAVAGE |
| 5287 | Training | NC | CITRIC ACID | 3000 | 5 | WATER | ORAL |
| | | | | | | | GAVAGE |
| 3313 | Training | NC | CLARITHROMYCIN | 476 | 5 | WATER | ORAL |
| | | | | | | | GAVAGE |
| 2719 | Training | NC | CLOTRIMAZOLE | 178 | 5 | CORN | ORAL |
| | | | | | | OIL | GAVAGE |
| 5656 | Training | NC | CORTISONE | 206 | 5 | CMC | ORAL |
| | | | | | | | GAVAGE |
| 6030 | Training | NC | CYCLOHEXIMIDE | 0.25 | 5 | WATER | ORAL |
| | | | | | | | GAVAGE |
| 5503 | Training | NC | DICHLORVOS | 17 | 5 | WATER | ORAL |
| | | | | | | | GAVAGE |
| 3348 | Training | NC | DICLOFENAC | 10 | 5 | CORN | ORAL |
| | | | | | | OIL | GAVAGE |
| 6609 | Training | NC | ERGOCALCIFEROL | 15 | 5 | CMC | ORAL |
| | | | | | | | GAVAGE |
| 3540 | Training | NC | ETHYLENE GLYCOL | 3525 | 5 | WATER | ORAL |
| | | | | | | | GAVAGE |
| 5668 | Training | NC | ETODOLAC | 24 | 5 | CMC | ORAL |
| | | | | | | | GAVAGE |
| 3320 | Training | NC | ETOPOSIDE | 188 | 5 | CORN | ORAL |
| | | | | | | OIL | GAVAGE |
| 3551 | Training | NC | FAMCICLOVIR | 1200 | 5 | SALINE | ORAL |
| | | | | | | | GAVAGE |
| 5411 | Training | NC | FLUOXETINE | 52 | 5 | CMC | ORAL |
| | | | | | | | GAVAGE |
| 3409 | Training | NC | FLUVASTATIN | 94 | 5 | CORN | ORAL |
| | | | | | | OIL | GAVAGE |
| 5362 | Training | NC | GENTIAN VIOLET | 18 | 5 | CORN | SUBCUTANEOUS |
| | | | | | | OIL | |
| 4654 | Training | NC | GLIMEPIRIDE | 2500 | 5 | CMC | ORAL |
| | | | | | | | GAVAGE |
| 4663 | Training | NC | GLIPIZIDE | 2500 | 5 | CMC | ORAL |
| | | | | | | | GAVAGE |
| 5433 | Training | NC | HEXACHLOROPHENE | 8 | 5 | CORN | SUBCUTANEOUS |
| | | | | | | OIL | |
| 6428 | Training | NC | HYDROCORTISONE | 56 | 5 | CORN | SUBCUTANEOUS |
| | | | | | | OIL | |
| 2697 | Training | NC | IBUPROFEN | 263 | 5 | CORN | ORAL |
| | | | | | | OIL | GAVAGE |
| 3523 | Training | NC | INDOMETHACIN | 12 | 5 | WATER | ORAL |
| | | | | | | | GAVAGE |
| 5395 | Training | NC | ISOPRENALINE | 15 | 5 | SALINE | INTRAVENOUS |
| 3450 | Training | NC | ISOTRETINOIN | 125 | 5 | CORN | ORAL |
| | | | | | | OIL | GAVAGE |
| 3212 | Training | NC | ITRACONAZOLE | 1093 | 5 | CORN | ORAL |
| | | | | | | OIL | GAVAGE |
| 2674 | Training | NC | KETOCONAZOLE | 227 | 3 | CORN | ORAL |
| | | | | | | OIL | GAVAGE |
| 6055 | Training | NC | KETOROLAC | 48 | 5 | WATER | ORAL |
| | | | | | | | GAVAGE |
| 3553 | Training | NC | LEAD(IV) ACETATE | 600 | 5 | SALINE | ORAL |
| | | | | | | | GAVAGE |
| 4524 | Training | NC | LEVAMISOLE | 120 | 5 | WATER | ORAL |
| | | | | | | | GAVAGE |
| 4541 | Training | NC | LIPOPOLYSACCHARIDE | 1.25 | 5 | SALINE | INTRAVENOUS |
| | | | E. COLI O55:B5 | | | | |
| 5810 | Training | NC | MEGESTROL ACETATE | 132 | 5 | CMC | ORAL |
| | | | | | | | GAVAGE |
| 3530 | Training | NC | MELOXICAM | 33 | 5 | CORN | ORAL |
| | | | | | | OIL | GAVAGE |
| 6437 | Training | NC | METHYLDOPA | 325 | 5 | WATER | ORAL |
| | | | | | | | GAVAGE |
| 3581 | Training | NC | N,N- | 1400 | 5 | SALINE | ORAL |
| | | | DIMETHYLFORMAMIDE | | | | GAVAGE |
| 3529 | Training | NC | NISOLDIPINE | 1125 | 5 | WATER | ORAL |
| | | | | | | | GAVAGE |
| 3297 | Training | NC | OMEPRAZOLE | 415 | 5 | CORN | ORAL |
| | | | | | | OIL | GAVAGE |
| 5533 | Training | NC | OXYQUINOLINE | 68 | 5 | CORN | SUBCUTANEOUS |
| | | | | | | OIL | |
| 5642 | Training | NC | PERGOLIDE | 1.1 | 5 | CMC | ORAL |
| | | | | | | | GAVAGE |
| 6364 | Training | NC | PERHEXILINE | 320 | 5 | CMC | ORAL |
| | | | | | | | GAVAGE |
| 4511 | Training | NC | PHENACETIN | 619 | 5 | CORN | ORAL |
| | | | | | | OIL | GAVAGE |
| 6091 | Training | NC | PIOGLITAZONE | 1500 | 5 | CORN | ORAL |
| | | | | | | OIL | GAVAGE |
| 5417 | Training | NC | PRAZIQUANTEL | 1200 | 5 | CMC | ORAL |
| | | | | | | | GAVAGE |
| 3305 | Training | NC | PROMETHAZINE | 113 | 5 | SALINE | ORAL |
| | | | | | | | GAVAGE |
| 5393 | Training | NC | PROPYLTHIOURACIL | 625 | 5 | CMC | ORAL |
| | | | | | | | GAVAGE |
| 5527 | Training | NC | PYRAZINAMIDE | 1500 | 5 | CMC | ORAL |
| | | | | | | | GAVAGE |
| 5677 | Training | NC | RABEPRAZOLE | 1024 | 5 | WATER | ORAL |
| | | | | | | | GAVAGE |
| 2950 | Training | NC | RALOXIFENE | 650 | 5 | CORN | ORAL |
| | | | | | | OIL | GAVAGE |
| 6438 | Training | NC | RIFABUTIN | 1500 | 5 | CMC | ORAL |
| | | | | | | | GAVAGE |
| 6375 | Training | NC | ROFECOXIB | 1550 | 5 | CORN | ORAL |
| | | | | | | OIL | GAVAGE |
| 2829 | Training | NC | ROSIGLITAZONE | 1800 | 5 | CORN | ORAL |
| | | | | | | OIL | GAVAGE |
| 5671 | Training | NC | ROXITHROMYCIN | 312 | 5 | CMC | ORAL |
| | | | | | | | GAVAGE |
| 3538 | Training | NC | SILDENAFIL | 420 | 5 | WATER | ORAL |
| | | | | | | | GAVAGE |
| 3261 | Training | NC | SPARFLOXACIN | 450 | 5 | CORN | ORAL |
| | | | | | | OIL | GAVAGE |
| 5460 | Training | NC | TICLOPIDINE | 223 | 5 | CMC | ORAL |
| | | | | | | | GAVAGE |
| 5544 | Training | NC | TOLAZAMIDE | 1500 | 5 | CMC | ORAL |
| | | | | | | | GAVAGE |
| 5328 | Training | NC | TRETINOIN | 7 | 5 | CORN | SUBCUTANEOUS |
| | | | | | | OIL | |
| 5568 | Training | NC | TRICHLOROACETIC ACID | 474 | 5 | CORN | SUBCUTANEOUS |
| | | | | | | OIL | |
| 6885 | Training | NC | TROGLITAZONE | 1200 | 5 | CORN | ORAL |
| | | | | | | OIL | GAVAGE |
| 6082 | Training | NC | VALPROIC ACID | 1500 | 5 | WATER | ORAL |
| | | | | | | | GAVAGE |
| 6143 | Training | NC | VINBLASTINE | 0.3 | 5 | SALINE | INTRAVENOUS |
| 4550 | Training | NC | ZIDOVUDINE | 1540 | 5 | CORN | ORAL |
| | | | | | | OIL | GAVAGE |

In Vivo Studies: Animals and Treatments

Male Sprague-Dawley (Crl:CD®(SD)(IGS)BR) rats (weight matched, 7 to 8 weeks of age and averaging 200 to 250 g) were purchased from Charles River Laboratories (Portage, Mich.) and housed individually in hanging, stainless steel, wire-bottom cages in a temperature (66-77° F.), light (12-hour dark/light cycle) and humidity (30-70%) controlled room. Water and Certified Rodent Diet #5002 (PMI Feeds, Inc, City, ST) were available ad libitum throughout the studies. Housing and treatment of the animals were in accordance with regulations outlined in the USDA Animal Welfare Act (9 CFR Parts 1, 2 and 3). Animals were assigned to groups such that mean body weights were within 10% of the mean vehicle control group. Test articles were administered either orally (10 ml of corn oil/kg body weight) or by intra-peritoneal injection (5 ml of saline/kg body weight), as indicated. Animals were dosed once daily starting on day 0, and necropsied 24 hrs after the last dose, as indicated. An equivalent number of time- and vehicle-matched control rats were treated concurrently.

Microarray Expression Profiling

Gene expression profiling, data processing and quality control were performed as previously described (see Example 1). Briefly, liver samples from 3 rats were chosen from each treatment and control group for gene expression analysis on the Codelink Uniset™ Rat 1 Bioarray (GE Healthcare Bio-Sciences Corp., Piscataway, N.J.). A complete list of probes on the array is provided on the Amersham Biosciences web site (www.amershambiosciences.com). Log transformed signal data for all probes were array-wise normalized used Array Qualifier (Novation Biosciences, Palo Alto, Calif.), a non-linear centralization normalization procedure adapted for the CodeLink microarray platform. Log₁₀ ratios for each experimental group are computed as the difference between the average of the logs of the normalized experimental signals and the average of the logs of the normalized control signals for each gene.

### Example 3

### Derivation of a Non-Genotoxic Hepatocarcinogenicity Signature

Overview

A linear classifier (i.e., gene signature) for non-genotoxic hepatocarcinogenicity was derived and optimized using liver expression profiles from rats treated with 25 in-class compound treatments shown to induce non-genotoxic hepatocarcinogenicity after 2 years of daily dosing, and 75 known not to induce non-genotoxic hepatocarcinogenicity under the dosing conditions used. More specifically, the SPLP algorithm was used to derive a linear classifier using data acquired as described in Examples 1 and 2 above to train and cross-validate.

Gene Signature Derivation

To derive the gene signature, a three-step process of data reduction, signature generation and cross-validation of the predictive signature was used. A total of 5443 gene probes from the total of 9911 on the CodeLink™ RU1 microarray were pre-selected based on having no missing values (e.g., invalid measurement or below signal threshold) in the positive class and less than 5% missing values in the negative class of the training set. Pre-selection of these genes increases the quality of the starting dataset but is not necessary in order to generate valid signatures according to the methods disclosed herein. These pre-selected genes are listed in Table 3 (submitted electronically herewith as ASCII formatted file “table3.txt” and incorporated by reference in its entirety). The genes listed in Table 3 (and Table 4 below) represent the sequences available in GenBank Release 155.0 (Aug. 16, 2006) and are identified by their GenBank accession number and UniGene title.

The signature used to predict onset of non-genotoxic hepatocarcinogenicity was derived using the A-SPLP linear programming algorithm as previously described (see U.S. Ser. No. 11/332,718, filed Jan. 12, 2006; and WO 2005/017807 A2, published Feb. 24, 2005, each of which is hereby incorporated by reference herein). Briefly, the SPLP algorithm finds an optimal linear combination of variables (i.e., gene expression measurements) that best separate the two classes of experiments in m dimensional space, where is equal to 5443.

The general form of this linear-discriminant based classifier is defined by n variables: x₁, x₂, . . . xₙ and n associated constants (i.e., weights): a₁, a₂, . . . aₙ, such that:

$$S = {{\sum }_{i}}^{n} {a}_{i} {x}_{i} - b$$

where S is the scalar product and b is the bias term. Evaluation of S for a test experiment across the n genes in the signature determines what side of the hyperplane in m dimensional space the test experiment lies, and thus the result of the classification. Experiments with scalar products greater than 0 are considered positive for non-genotoxic hepatocarcinogenicity.

Signature Validation

Cross-validation provides a reasonable approximation of the estimated performance on independent test samples. The signature was trained and validated using a split sample cross validation procedure. Within each partition of the data set, 60% of the positives and 40% of the negatives were randomly selected and used as a training set to derive a unique signature, which was subsequently used to classify the remaining test cases of known label. This process was repeated 20 times, and the overall performance of the signature was measured as the percent true positive and true negative rate averaged over the 20 partitions of the data set, which is equivalent to testing 392 samples.

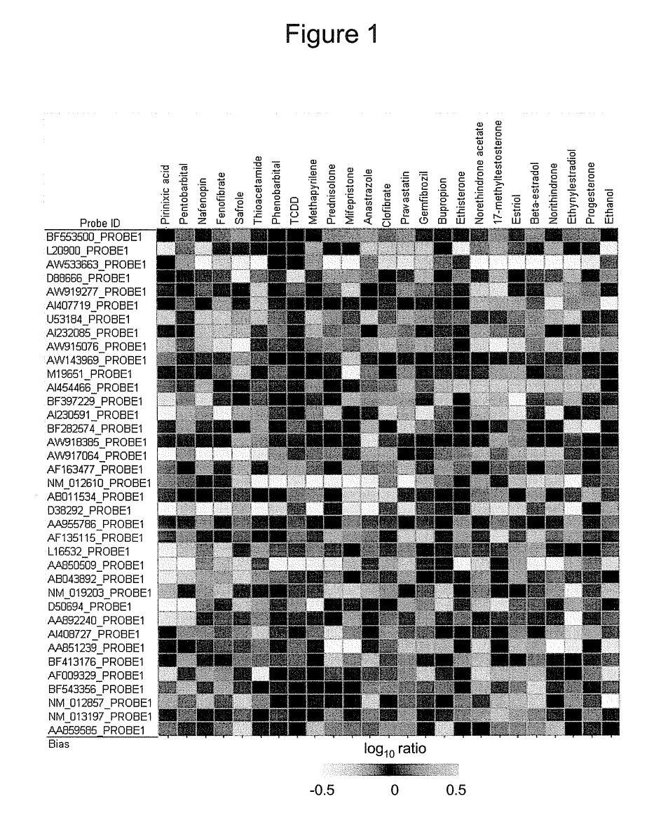

Based on split sample (60% training, 40% test, 20 random splits) cross validation, the signature has an estimated sensitivity of 56%, a specificity of 94%, and a log odds ratio of 2.92. Repeated randomization and classification of the 100 compound training set resulted in a mean log odds ratio of −0.007±0.331 (n=100), which is significantly different from the true training set (p<2.2e-16) indicating the estimated accuracy is not due to chance. The complete signature generated using 100% of the training data consisted of 37 probes and their corresponding weights (FIG. 1).

Results

Using 5443 pre-selected genes whose accession numbers are listed in Table 3 (submitted electronically herewith as ASCII formatted file “table3.txt” and incorporated by reference in its entirety), the A-SPLP algorithm was trained to produce a gene signature for non-genotoxic hepatocarcinogenicity comprising 37 genes, their associated weights and a bias term that perfectly classified the training set. The 37 genes and the parameters of the signature are listed below in Table 4 and the Sequence Listing (submitted electronically herewith as ASCII formatted file “sequence.txt” and incorporated by reference in its entirety).

TABLE 4
37 Gene Signature for Non-Genotoxic Hepatocarcinogenicity

| | SEQ | | | Average | Average |
|---|---|---|---|---|---|
| GenBank | ID | | Weight | Log₁₀ | Impact |
| Accession | NO. | UniGene Title | (w) | Ratio (r) | (w × r) |
| BF553500 | 1 | Cbp/p300-interacting transactivator 4 | 0.90 | 0.64 | 0.5773 |
| L20900 | 2 | islet cell autoantigen 1 | 1.32 | 0.28 | 0.3684 |
| AW533663 | 3 | proline dehydrogenase | −1.05 | −0.29 | 0.3092 |
| D88666 | 4 | phosphatidylserine-specific phospholipase A1 | −1.35 | −0.16 | 0.2156 |
| AW919277 | 5 | Ca2+-dependent activator for secretion protein 2 | −0.85 | −0.21 | 0.1804 |
| AI407719 | 6 | ubiquitin specific peptidase 2 | −0.64 | −0.22 | 0.1390 |
| U53184 | 7 | LPS-induced TN factor | 0.71 | 0.13 | 0.0893 |
| AI232085 | 8 | TRNA nucleotidyl transferase, CCA-adding, 1 | −0.86 | −0.10 | 0.0826 |
| AW915076 | 9 | G protein-coupled receptor 146 | −1.32 | −0.06 | 0.0797 |
| AW143969 | 10 | EST | 0.87 | 0.06 | 0.0518 |
| M19651 | 11 | fos-like antigen 1 | 0.44 | 0.08 | 0.0334 |
| AI454466 | 12 | EST | 0.23 | 0.12 | 0.0274 |
| BF397229 | 13 | protocadherin 19 (Hs.) | −0.88 | −0.03 | 0.0273 |
| AI230591 | 14 | ctla-2-beta protein (141 AA) | −0.25 | −0.10 | 0.0248 |
| BF282574 | 15 | growth hormone receptor | 1.53 | 0.02 | 0.0237 |
| AW918385 | 16 | prickle-like 1 (Drosophila) | −1.09 | −0.02 | 0.0221 |
| AW917064 | 17 | EST | −0.08 | −0.29 | 0.0218 |
| AF163477 | 18 | chemokine (C-C motif) ligand 22 | −0.17 | −0.11 | 0.0200 |
| NM_012610 | 19 | nerve growth factor receptor (TNFR superfamily, | 0.26 | 0.06 | 0.0170 |
| | | member 16) | | | |
| AB011534 | 20 | EGF-like-domain, multiple 4 | 0.20 | 0.06 | 0.0130 |
| D38292 | 21 | protein tyrosine phosphatase, receptor type, R | −0.24 | −0.05 | 0.0115 |
| AA955786 | 22 | A kinase (PRKA) anchor protein 5 | −0.22 | −0.03 | 0.0071 |
| AF135115 | 23 | cullin 5 | −0.30 | −0.02 | 0.0062 |
| L16532 | 24 | cyclic nucleotide phosphodiesterase 1 | 0.10 | 0.06 | 0.0061 |
| AA850509 | 25 | thyroid hormone receptor interactor 13 | 0.20 | 0.03 | 0.0060 |
| AB043892 | 26 | calsenilin, presenilin binding protein, EF hand | −0.19 | −0.03 | 0.0058 |
| | | transcription factor | | | |
| NM_019203 | 27 | testis specific X-linked gene | 0.04 | 0.13 | 0.0047 |
| D50694 | 28 | proteasome (prosome, macropain) 26S subunit, | 0.04 | 0.09 | 0.0037 |
| | | ATPase 2 | | | |
| AA892240 | 29 | EST | 0.21 | 0.00 | −0.0003 |
| AI408727 | 30 | EST | −0.03 | 0.04 | −0.0013 |
| AA851239 | 31 | exostoses (multiple)-like 2 | 0.12 | −0.04 | −0.0045 |
| BF413176 | 32 | EST | 0.13 | −0.04 | −0.0047 |
| AF009329 | 33 | basic helix-loop-helix domain containing, class B3 | −0.12 | 0.10 | −0.0122 |
| BF543356 | 34 | histidine rich calcium binding protein | 0.37 | −0.07 | −0.0263 |
| NM_012857 | 35 | lysosomal membrane glycoprotein 1 (Non-specific | −1.00 | 0.03 | −0.0299 |
| | | probe) | | | |
| NM_013197 | 36 | aminolevulinic acid synthase 2 | 0.23 | −0.38 | −0.0852 |
| AA859585 | 37 | EST | −0.75 | 0.14 | −0.1030 |
| Bias | | | 0.71 | | |

Average impact represents the contribution of each gene towards the scalar product, and is calculated as the product of the average log₁₀ ratio and the weight calculated across the 25 compounds in the positive class listed in Table 2.

As shown in Table 4, the genes are ranked in descending order of percent contribution, which is calculated as the fraction of the average positive impact each gene in the positive training class has relative to the sum of all positive impacts. Genes with a negative average impact are considered penalty genes.

As shown in FIG. 1, the expression log 10 ratio of each gene was plotted in the depicted “heat map” across all 25 in-class compound treatments in the training set. The sum of the impact across all 37 genes for each treatment, and the resulting scalar product are presented along the two rows below the plot. The bias term for the 37 gene signature was 0.71.

Cross Validation

In order to validate the 37 gene signature on independent test samples, the remaining 21 NGTC chemicals and 26 NC chemicals were profiled on days 1, 3 and 5 as described above and evaluated with the signature. Experiments with a scalar product greater than zero are classified as non-genotoxic hepatocarcinogens. To account for compound specific toxicodynamics and toxicokinetics, the maximum scalar product observed over the multiple time points for each compound was used to classify each compound as being positive or negative. This increases the sensitivity of the signature with little loss of specificity. As shown below in Table 5, the signature correctly predicted 18 of the 21 (85.7%) NGTC test chemicals as being hepatocarcinogens, the majority of which exhibited the maximum scalar product on day 5.

TABLE 5

| | Dose | | | | | | | | |
|---|---|---|---|---|---|---|---|---|---|
| | (mg/kg/d) | Vehicle | Route | Class | Day 1 | Day 3 | Day 5 | Max | Result |
| Compound Name | | | | | | | | | |
| ACETAMINOPHEN | 972 | CORN OIL | PO | NGTC | 0.33 | −0.11 | 0.77 | 0.77 | TP |
| BETA- | 1500 | CMC | PO | NGTC | 0.81 | 0.56 | 0.60 | 0.81 | TP |
| NAPHTHOFLAVONE | | | | | | | | | |
| BEZAFIBRATE | 617 | CORN OIL | PO | NGTC | 0.34 | 2.72 | 2.38 | 2.72 | TP |
| BIS(2- | 1000 | CORN OIL | PO | NGTC | 0.47 | 1.04 | −0.09 | 1.04 | TP |
| ETHYLHEXYL)PHTHALATE | | | | | | | | | |
| CARBAMAZEPINE | 490 | CMC | PO | NGTC | −1.90 | −1.17 | −0.35 | −0.35 | FN |
| CARBIMAZOLE | 400 | WATER | PO | NGTC | −0.72 | −0.38 | −1.34 | −0.38 | FN |
| CARBON | 1175 | CORN OIL | PO | NGTC | −0.15 | 0.41 | 0.50 | 0.50 | TP |
| TETRACHLORIDE | | | | | | | | | |
| CHLOROFORM | 600 | CORN OIL | PO | NGTC | 1.50 | 0.74 | 0.27 | 1.50 | TP |
| CLOFIBRIC ACID | 448 | CORN OIL | PO | NGTC | −0.37 | 0.86 | 0.63 | 0.86 | TP |
| CYPROTERONE | 2500 | CMC | PO | NGTC | 0.80 | 1.16 | 0.44 | 1.16 | TP |
| ACETATE | | | | | | | | | |
| DIETHYLSTILBESTROL | 280 | CORN OIL | PO | NGTC | 2.75 | 2.29 | 1.46 | 2.75 | TP |
| ETHYLESTRENOL | 390 | CMC | PO | NGTC | 0.99 | 2.25 | 0.50 | 2.25 | TP |
| FENBENDAZOLE | 375 | CORN OIL | PO | NGTC | −0.19 | 0.17 | −0.89 | 0.17 | TP |
| FLUCONAZOLE | 394 | CORN OIL | PO | NGTC | 0.28 | 0.57 | 3.63 | 3.63 | TP |
| LOVASTATIN | 1500 | CORN OIL | PO | NGTC | 0.74 | 1.07 | 0.78 | 1.07 | TP |
| OXFENDAZOLE | 1500 | CMC | PO | NGTC | −1.22 | −0.74 | 0.32 | 0.32 | TP |
| OXYMETHOLONE | 1170 | CMC | PO | NGTC | −0.36 | −0.62 | −0.98 | −0.36 | FN |
| SIMVASTATIN | 1200 | CORN OIL | PO | NGTC | 0.50 | 1.60 | | 1.60 | TP |
| SPIRONOLACTONE | 300 | CMC | PO | NGTC | −0.42 | 0.15 | 0.37 | 0.37 | TP |
| STANOZOLOL | 150 | WATER | PO | NGTC | 0.54 | 0.14 | | 0.54 | TP |
| TESTOSTERONE | 375 | CMC | PO | NGTC | 0.25 | 0.45 | −0.63 | 0.45 | TP |
| Compound_Name | | | | | | | | | |
| 1,1-DICHLOROETHENE | 600 | WATER | PO | NC | −1.01 | −1.36 | −0.80 | −0.80 | TN |
| AMOXAPINE | 313 | CMC | PO | NC | −1.00 | −1.48 | −1.55 | −1.00 | TN |
| ATORVASTATIN | 300 | CORN OIL | PO | NC | −0.30 | 1.85 | 1.40 | 1.85 | FP |
| BENZETHONIUM | 30 | CORN OIL | SC | NC | −0.13 | −1.34 | −1.68 | −0.13 | TN |
| CHLORIDE | | | | | | | | | |
| CHOLECALCIFEROL | 8 | CMC | PO | NC | −1.38 | −1.52 | −2.86 | −1.38 | TN |
| CITALOPRAM | 90 | CORN OIL | PO | NC | −0.78 | −0.60 | −0.28 | −0.28 | TN |
| CLOMIPHENE | 250 | CMC | PO | NC | −1.72 | −1.71 | −2.14 | −1.71 | TN |
| CLOMIPRAMINE | 115 | WATER | PO | NC | −1.46 | −1.93 | −2.40 | −1.46 | TN |
| CYCLOSPORIN A | 350 | CORN OIL | PO | NC | −0.41 | −0.57 | −0.18 | −0.18 | TN |
| CYTARABINE | 487 | SALINE | IV | NC | −0.71 | −1.25 | −1.84 | −0.71 | TN |
| DIAZEPAM | 710 | CMC | PO | NC | −1.40 | −0.06 | 0.52 | 0.52 | FP |
| ERYTHROMYCIN | 1500 | CMC | PO | NC | −1.36 | −1.18 | −1.32 | −1.18 | TN |
| FINASTERIDE | 800 | CORN OIL | PO | NC | 0.10 | 0.22 | −0.16 | 0.22 | FP |
| GERANIOL | 1500 | CMC | PO | NC | −0.42 | −0.30 | −1.24 | −0.30 | TN |
| LORAZEPAM | 2000 | CMC | PO | NC | −1.47 | −0.24 | 0.10 | 0.10 | FP |
| NEVIRAPINE | 200 | SALINE | PO | NC | −0.50 | −0.80 | −0.38 | −0.38 | TN |
| OLANZAPINE | 23 | CMC | PO | NC | −0.58 | −0.56 | −0.54 | −0.54 | TN |
| PEMOLINE | 70 | CMC | PO | NC | −1.57 | −1.80 | −1.86 | −1.57 | TN |
| PHENOTHIAZINE | 386 | CORN OIL | PO | NC | −0.89 | 0.70 | −0.67 | 0.70 | FP |
| PRIMIDONE | 750 | CMC | PO | NC | −1.01 | −1.00 | −0.15 | −0.15 | TN |
| PROPYLENE GLYCOL | 2000 | WATER | PO | NC | −0.80 | −1.25 | −1.01 | −0.80 | TN |
| QUETIAPINE | 500 | CMC | PO | NC | −1.20 | −0.42 | −1.23 | −0.42 | TN |
| TETRACYCLINE | 1500 | WATER | PO | NC | −0.40 | −1.18 | −1.31 | −0.40 | TN |
| TOCAINIDE | 224 | CORN OIL | PO | NC | −1.63 | −1.26 | −0.91 | −0.91 | TN |
| VENLAFAXINE | 320 | WATER | PO | NC | −0.95 | −2.09 | −1.51 | −0.95 | TN |
| VINORELBINE | 1.5 | SALINE | IV | NC | −1.94 | −1.74 | −1.37 | −1.37 | TN |

| | Dose | | | | | | | |
|---|---|---|---|---|---|---|---|---|
| Compound_Name | (mg/kg/d) | Vehicle | Route | Class | Day 1 | Day 3 | Day 5 | Max |
| 2- | 30 | CMC | PO | GTC | −0.60 | 0.03 | −0.22 | 0.03 |
| ACETYLAMINOFLUORENE | | | | | | | | |
| HYDRAZINE | 45 | WATER | PO | GTC | −0.10 | −1.31 | −1.48 | −0.10 |
| AFLATOXIN B1 | 0.3 | CMC | PO | GTC | −0.63 | −1.22 | −0.79 | −0.63 |
| N- | 34 | SALINE | PO | GTC | −2.17 | −1.83 | −1.41 | −1.41 |
| NITROSODIETHYLAMINE | | | | | | | | |

| | | Dose | | | | | | | |
|---|---|---|---|---|---|---|---|---|---|
| Compound_Name | Gender | (mg/kg/d) | Vehicle | Route | Day 1 | Day 3 | Day 5 | Max | Result |
| BETA-ESTRADIOL 3- | Female | 25 | CORN | SC | 0.81 | 0.25 | 0.03 | 0.81 | TP |
| BENZOATE | | | OIL | | | | | | |
| NORETHINDRONE | Female | 75 | CORN | PO | 1.61 | 1.38 | 2.03 | 2.03 | TP |
| | | | OIL | | | | | | |
| RALOXIFENE | Female | 650 | CORN | PO | −0.66 | −1.25 | −2.11 | −0.66 | TN |
| | | | OIL | | | | | | |

Of the 26 NC test chemicals, 21 (80.7%) were correctly predicted negative. Although the training set is composed of mainly small molecule therapeutics (88%), the signature has broad utility across diverse structural and mechanistic classes, including industrial toxicants and environmental pollutants. For example, the signature correctly predicted the hepatotoxicants chloroform and carbon tetrachloride as positive, and 1,1-dichloroethene and propylene glycol as negative. In addition to the 47 test chemicals, the signature predicted 3 genotoxic hepatocarcinogens (aflatoxin B1, hydrazine, and N-nitrosodiethylamine) as negative, and 2-acetylaminofluorene as weakly positive (SP=0.03) (Table 4), thus indicating that the signature is specific for chemicals that induce hepatocarcinogenicity through non-genotoxic mechanisms, as expected given the composition of the training set.

### Example 4

### Stripping of Non-Genotoxic Hepatocarcinogenicity Signatures to Produce a Necessary Set of Genes

In order to understand the biological basis of classification and provide a subset of genes useful in alternative signatures for non-genotoxic hepatocarcinogenicity, an iterative approach is taken in order to identify all the genes that are necessary and sufficient to classify the training set.

Starting with the 5443 pre-selected genes on the Codelink RU1 microarray (see Table 3), a signature for non-genotoxic hepatocarcinogenicity is generated with the SPLP or A-SPLP algorithm and cross-validated using multiple random partitions (60% training: 40% test) of the data set, as described above in Examples 2 and 3. The 37 genes identified previously in the first signature (see Table 4) as being sufficient to classify the training set are removed and the algorithm repeated to identify derive additional gene signatures with increasingly lower LOR. This is repeated until the test LOR of the gene signature drops below a pre-selected performance threshold, or reaches zero. The assembly of genes from each gene signature with a performance above the LOR threshold constitutes the “necessary set” for non-genotoxic hepatocarcinogenicity. That is, these gene are necessary to classify the training set with a test LOR the average LOR achieved with random label sets. The necessary set identifies a reasonable number of genes with a demonstrated ability to uniquely discriminate in-class compound treatments with an approximate accuracy above a set threshold.

### Example 5

### Using a Necessary Set to Generate New Signatures for Non-Genotoxic Hepatocarcinogenicity Based on Random Selection of High Impact Gene Subsets

As shown above in Examples 1-3, a predictive signature for non-genotoxic hepatocarcinogenicity comprising 37 genes may be derived using gene expression data from a microarray in the context of a chemogenomic database. Using the signature stripping method described above in Example 4, additional high performing predictive signatures for non-genotoxic hepatocarcinogenicity may also be derived wherein each of the signatures is non-overlapping, i.e., comprises genes not used in any of the other signatures. Together, the union of the genes in these five signatures comprises a set of genes that is necessary for deriving a predictive signature for non-genotoxic hepatocarcinogenicity capable of classifying the training set above a selected threshold LOR level.

This example demonstrates that additional signatures for non-genotoxic hepatocarcinogenicity may be generated based on the necessary set of genes. In addition, it is shown that a number of genes must be selected from the necessary set in order to generate a signature for non-genotoxic hepatocarcinogenicity capable of performing above a selected threshold LOR.

For each gene from the necessary set an impact factor is calculated, corresponding to the product of the gene's weight and the gene's expression mean log₁₀ ratio in the positive class (i.e., non-genotoxic hepatocarcinogens). Subsets of genes are chosen randomly from the necessary set of so that the sum of the impacts of all genes in the subset accounted for 1, 2, 4, 8, 16, 32, or 64% of the total impact. Total impact is defined as the sum of the individual impacts of all genes in the necessary set. This random subset selection procedure is repeated to general a plurality of gene subsets. Each of these randomly selected plurality of subsets is used as input to compute a non-genotoxic hepatocarcinogenicity signature using the SPLP algorithm as described in Example 3 above. A training LOR and a 10-fold cross-validated test LOR are calculated for each signature.

Using this method it is possible to use the necessary set to generate custom gene signatures for non-genotoxic hepatocarcinogenicity that use a minimal number of genes to perform at a selected threshold LOR.

For example, it may be determined that signatures for non-genotoxic hepatocarcinogenicity capable of performing with an average training LOR of 2.50 may be generated starting with random subsets selected from the necessary that have an average of only 10 genes that together have only 2% of the total impact of the necessary set. Alternatively, if desired comparably higher performing signatures may be derived from the necessary set by selecting random subsets having a percent impact of 8% or higher.

### Example 6

### Functional Characterization of the Necessary Set of Genes for Non-Genotoxic Hepatocarcinogenicity by Random Supplementation of a Fully Depleted Set

This example illustrates how the necessary set of genes for classifying non-genotoxic hepatocarcinogenicity may be functionally characterized by randomly supplementing and thereby restoring the ability of a depleted gene set to generate non-genotoxic hepatocarcinogenicity signatures capable of performing on average above a threshold LOR. In addition to demonstrating the power of the information rich genes in the non-genotoxic hepatocarcinogenicity necessary set, this example illustrates a system for describing any necessary set of genes in terms of its performance parameters.

As described in Example 4, a necessary set of genes for the non-genotoxic hepatocarcinogenicity classification question is generated via the stripping method. In the process, a corresponding fully depleted set of genes (i.e., the full dataset of 5443 genes minus the number of genes in the necessary set) is also generated. This fully depleted set of genes is not able to generate an non-genotoxic hepatocarcinogenicity signature capable of performing with a LOR greater than or equal to pre-selected LOR threshold used in generating the necessary set.

A subset of randomly selected genes (or the same number as the necessary set) are removed from the fully depleted set. Then a randomly selected set including 10, 20, 40 or 80% of the genes from either: (a) the necessary set; or (b) the set of randomly removed from the fully depleted set; is added back to the depleted set. The resulting “supplemented” depleted set is then used to generate an non-genotoxic hepatocarcinogenicity signature, and the performance of this signature is cross-validated as described in Example 3. This process is repeated 20 times for each of the different percentage supplementations of genes from the necessary set and the random subset of genes removed from the original depleted set. Twenty cross-validated non-genotoxic hepatocarcinogenicity signatures are obtained for each of the various percentage supplementations of the depleted set. Average LOR values are calculated based on the 20 signatures generated for each percentage supplementation.

The results demonstrate how supplementation with a percentage of randomly selected genes from the non-genotoxic hepatocarcinogenicity necessary set effectively “revives” the performance of a fully depleted set for generating classifiers. Thus, the non-genotoxic hepatocarcinogenicity necessary set of genes may be functionally characterized as the set of genes for which a randomly selected percentage can be used to supplement a set of genes fully depleted for non-genotoxic hepatocarcinogenicity classification (i.e., not capable of producing non-genotoxic hepatocarcinogenicity signatures above the pre-selected avg LOR threshold), such that the resulting “revived” gene set is able to generate a non-genotoxic hepatocarcinogenicity signatures with an average LOR greater than or equal to the threshold.

### Example 7

### Construction and Use of a DNA Array for Predicting Non-Genotoxic Hepatocarcinogenicity

The subset of genes identified to be necessary to classify the non-genotoxic hepatocarcinogenicity training set listed in Table 2 may be used as the basis for a DNA array diagnostic device for predicting non-genotoxic hepatocarcinogenicity. The device may be used in a therapeutic monitoring context, such as for monitoring the response of an individual to a compound that is suspected of possibly causing non-genotoxic hepatocarcinogenicity (or related hepatotoxic side effects). Alternatively, smaller sufficient subsets of genes the necessary set, which may be selected according to the methods of Examples 4 and 5 described above, may be used as the basis for a DNA array.

The probe sequences used to represent the necessary set genes on the array may be the same ones used on the Amersham CodeLink™ RU1 platform DNA array used to derive the non-genotoxic hepatocarcinogenicity signature as described in Examples 1-3. The probes are pre-synthesized in a standard oligonucleotide synthesizer and purified according to standard techniques. The pre-synthesized probes are then deposited onto treated glass slides according to standard methods for array spotting. For example, large numbers of slides, each containing the set of probes, are prepared simultaneously using a robotic pen spotting device as described in U.S. Pat. No. 5,807,522. Alternatively, the necessary set probes may be synthesized in situ one or more glass slides from nucleoside precursors according to standard methods well known in the art such as ink-jet deposition or photoactivated synthesis.

The DNA probe arrays made according to this method are then each hybridized with a fluorescently labeled nucleic acid sample. The nucleic acid may be derived from mRNA obtained from a biological fluid (e.g., blood) or a tissue sample from a compound treated individual. Any of the well-known methods for preparing labeled samples for DNA probe array hybridization may be used. The fluorescence intensity data from hybridization of the sample to the DNA array of necessary set (or fewer) genes of the necessary set is used to calculate expression log ratios for each of the genes. Depending on the specific gene signature selected for use in predicting non-genotoxic hepatocarcinogenicity (e.g., the genes in iteration 1 of Table 4), the scalar product for that signature is calculated (i.e., sum of the products of expression log₁₀ ratio and weight for each gene less the bias). If the scalar product is greater than zero then the sample is classified as positive (i.e., onset of non-genotoxic hepatocarcinogenicity is predicted).

### Example 8

### Construction and Use of a DNA Array for Non-Genotoxic Hepatocarcinogenicity Using Human Orthologs of Rat Genes

The set of rat genes identified in the necessary set to classify non-genotoxic hepatocarcinogenicity may be used to construct a DNA array diagnostic device for predicting the non-genotoxic hepatocarcinogenicity in humans. The human orthologs for the necessary set genes may be obtained from well-known public databases.

The necessary set of human orthologs may be used to derive additional signatures for non-genotoxic hepatocarcinogenicity according to the methods of Example 5. A DNA microarray may then be fabricated using polynucleotides corresponding to the human genes or any subset thereof. The device may be used in a therapeutic monitoring context, such as for monitoring the response of an individual to a compound that is suspected of possibly causing non-genotoxic hepatocarcinogenicity or related toxic effects.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

What is claimed:

1. A method for testing whether a compound will induce non-genotoxic hepatocarcinogenicity in a test subject, the method comprising:
a) administering a dose of compound to at least one test subject;
b) after a selected time period, obtaining a biological sample from the at least one test subject;
c) measuring the expression levels in the biological sample of at least a plurality of genes selected from Table 4; and
d) determining whether the sample is in the positive class for non-genotoxic hepatocarcinogenicity using a linear classifier comprising at least the plurality of genes for which the expression levels are measured.

2. The method of claim 1, wherein determining whether the sample is in the positive class comprises determining a scalar product based on the sum of the products of each gene's expression log₁₀ ratio and weight, and subtracting the bias, wherein a positive sum indicates the sample is in the positive class.

3. The method of claim 1, wherein the test subject is a mammal selected from the group consisting of cat, dog, monkey, mouse, pig, rabbit, and rat.

4. The method of claim 1, wherein the biological sample comprises liver tissue.

5. The method of claim 1, wherein the test subject is selected from the group consisting of a cell culture and a tissue culture.

6. The method of claim 1, wherein the selected period of time is about 5 days or fewer.

7. The method of claim 1, wherein the expression levels are measured as log₁₀ ratios of the compound-treated biological sample to a compound-untreated biological sample.

8. The method of claim 1, wherein the linear classifier comprises the genes and weights corresponding to the gene signature listed in Table 4.

9. The method of claim 11, wherein the linear classifier for non-genotoxic hepatocarcinogenicity classifies the in-class versus not in-class compounds listed in Table 2 with a training log odds ratio of greater than or equal to 2.50.

10. A reagent set for testing whether non-genotoxic hepatocarcinogenicity will occur in a test subject comprising a plurality of polynucleotides or polypeptides representing a plurality of genes selected from Table 4.

11. The reagent set of claim 12, wherein the plurality of genes are selected from a linear classifier capable of classifying non-genotoxic hepatocarcinogenicity with a training log odds ratio of greater than or equal to 2.50.

12. The reagent set of claim 12, wherein the plurality of genes is the set of 37 genes in Table 4.

13. The reagent set of claim 12, wherein the reagents are polynucleotide probes capable of hybridizing to the plurality of genes selected from Table 4.

14. The reagent set of claim 15, wherein the polynucleotide probes are primers for amplification of the plurality of genes.

15. The reagent set of claim 15, wherein the polynucleotide probes are immobilized on one or more solid surfaces.

16. The reagent set of claim 12, wherein the reagents are polypeptides that bind to a plurality of proteins encoded by the plurality of genes selected from Table 4.

17. The reagent set of claim 18, wherein the proteins are secreted proteins.

18. An apparatus for predicting whether non-genotoxic hepatocarcinogenicity will occur in a test subject comprising a reagent set according to claim 12.

19. The apparatus of claim 20, wherein the reagents are polynucleotides.

20. The apparatus of claim 20, wherein the reagents are polypeptides.


SEQUENCE LISTING (37 sequences)

SEQ ID NO: 1 - DNA, 2094 bp, Rattus norvegicus
ggcacgagctccgatctccgctgagaggctcctgggggccggggctccgaggaaaatggttcgatgttattaaaaatgaatcttaagaagaaaaatgaatcacagcagttaaactatggagttctgctacttgtaagaagtggagaagcctgagattacatcactgacccttgatcctccactgagctaaaacaccagctggtaattgcctatgattttatagacttccctccatctgctgggtccaagtgtccgtctgactgctctggtaccggagcatctttatttctgcatctaaacttgtaaaaagcacatcgaatcttgttccccaggagaaaatcttcaatgtaaccattttcaatgtatccgatgatacaagcgcattgtaatctccaggtagaagcagctttatcagtggaaagggtttaatagaacatatcctatcatgctttttctctgccccttctcaaatcatcagcagtagaaaagagaagaaaacatgtcaggacacaaatgttattcctgggagttgcaggatcggtttgctcaagataagtcagttgtcaataagatgcaacagaaatattgggaaacgaatgaggcctttatcaaagccacagggaagaaggaagatgaacatgtcgttgcttctgatgcagacctggatgccaagctagagctgtttcattcgattcagagaacctgtttggacttgtctaaagcaattgtgctctatcaaaagagaatatgtttcttgtctcaagaagaaaatgaactgggaaaatttctccgatcccaaggcttccaggacaaaacccgagcaggaaaaatgatgcaagccacaggaaaggccctctgcttttcctcccagcaaaggttggccttgagaaaccctttgtgtcgatttcaccaagaagtagagacttttagacatcgggccatctccgatacctggctgacagtgaaccgcatggagcagtgccggacagaatatagaggggcgttattgtggatgaaggacgtgtctcaggaactggatccagacctctacaagcaaatggagaagttcaggaaggtacagacacaagtccgcctcgcgaagaagaactttgacaaattgaagatggatgtgtgtcaaaaggtggatcttcttggagcaagcagatgtaacctcttatctcacatgctagcaacatatcagaccactctgctccacttttgggagaaaacttctcacaccatggcagccatccatgagagcttcaaaggctatcaaccatatgaattcacaacgttaaagagcttacaagaccctatgaaaaagctagtcgagaaagaaaagaagaagagctcccggagggaaaaccgggaggctgtggcacaggagccgaggcagttaatttcattggaggaagagaaccagcacaaagaatcctctacttgccagaaggaggagggaaaaagcgttccgtcgtctgtagacaagagttctgcagatgatgcatgctcaggacccatagatgaactattagacgtgaaacctgaggaagcttgcctgggtcccatggcagggaccccagaacctgaaagtggggacaaggacgacctcctgctgttgaacgagatcttcagcacttccagcctggatgaaggggagttcagcagggagtgggctgcggtgttcggagacgaccggctgaaggaaccagcccccatgggggcccagggagagccagaccccaagccccagataggctctgcgttccttccttcacagcttttagaccaaaacatgaaagatctccaggcctctctccaagagcctgccaaggctgcctcggacctgactgcctggttcagcctctttgctgacctcgaccccctatcaaatcctgatgctattgggaaaaccgataaagaacacgaattgctcaatgcatgagtctgcaaccttcaacaggggagccctccggccactccgcaacacctcatccagggcttgcagaagtctaacgtgctcagtacgctgttttaatatttacatgccattttaataaaacgagagggtcaaggccctgtttctatcgctataaa

SEQ ID NO: 2 - DNA, 1743 bp, Rattus norvegicus
ctccagcccagcgatgtgtcctggcctctgggggacatgcttctggttgtggggatcacttttatggctcagcattggaagatcagggaacgtaccccctaccacccaaccgaagtgcactgacttccagagtgccaacctcctcagaggcaccaacctcaaagtccagtttctcctctttaccccctcggaccccggctgtggacaactagtagaagaggacagtgacatccggaactctgagttcaatgccagtctgggaaccaaactaattattcatggattcagggcattaggaacaaaaccttcttggatcaacaagtttatcagagctctcctgcgggcagcggatgctaatgtgattgcagtggactgggtttatggttccacgggcatgtacttctcagctgtggagaatgtggtcaagttgagcctggagatctcccgtttcctcagcaaacttttggagctgggtgtgtcagagtcctcaatccacatcattggtgtcagtctgggggctcatgttggaggcatggtggggcatttctacaaaggccagttgggacggatcacaggtctggatcctgctggaccagagtacaccagagccagcctggaggaacgcttggattctggagatgccctgtttgtggaagccatccacacagacactgacaatttgggtatccggattcctgtcggacatgtggactactttgtcaatggaggccaagaccagcctggatgccctgcattcattcacgcaggttacagttacttgatctgtgatcacatgagggctgtacatctctatatcagtgccttggagaacacttgcccactgatggcctttccctgtgccagctacaaggccttccttgcaggagactgtctggactgctttaaccctttcctgctctcctgtccgaggattggactggtggaacgaggtggtgtcaagattgagccgctccccaaggaagtgagggtctatctccagactacatccagtgccccatactgtgtgcaccacagcctcgtggagtttaatttgaaggagaagagaaaaaaggataccagcatcgaggtcacctttcttggcaacaatgtaacgtcctcggtcaagatcaccatacctaaagatcaccttgaagggagagggatcatcgcccatcaaaacccacactgccagataaaccaggtgaagctcaagttccacatttctagccgggtttggagaaaagacaggactcccattgttgggactttctgtaccgctcctctgccagtcaatgacagcaagaagacggtctgcatccctgagccagtgcgtctgcaagtgagcatggctgttctccgggacctgaaaatggcctgtgtgtagcctgagcctactcttgaggcagaggccggaatttttcgagggcagtgtggcaagggctgtttgcaagcgccatattctaccctgtttctactaagggggggaaggccaaattcttggtggttttctccataagtagttactgtggaagggacaggtgactcatattacagaacttgatctccgtcaccgacttacaaagctttatacagatgccatttcagcttctctatttcaacacaactgtgattgcctcacagccttaagtatctatacttaggattcaatggaaaatgtactcggagaaatgttttaaataaattgtcatggaatatctgaaaaaaaaaaaaaaagaaaaaaaa

SEQ ID NO: 3 - DNA, 2006 bp, Rattus norvegicus
gcgcgcgcggccagcgagacctcccggctccactggcggcgcgggcggcggtaaaatgtcggctccaggaccttaccaagcagctgcaggcccttctgtaatgcctactgcacccccaacctatgaagaaacagtgggtgtcaacagttactacccaacgcccccagcacctcagccgggaccagccacagggctcattacgggcccggatgggaagggcatgaatccaccttcgtactacacccagcccgtgcctgtccccaacgccaacgcaattgcagtgcagacggtttatgtgcagcagcccatctccttctacgaccgccccatccagatgtgctgtccttcctgcaacaagatgatcgtgacccagttgtcctacaatgcaggagccctcacctggctctcctgtggcagtctgtgtctgctgggatgcgtcgctggctgctgcttcattccgttttgcgtggacgccctgcaggatgtggaccattactgccccaactgcaaagcgctcctgggcacctacaaacgtttgtaggactcggccagccacaggggaggtagacaagtgctcccggaagtcacccaacccttgcccagcgcagccagaagacgtcccttccttgatctcttcatgagctcgtcttcgtgtcttttagggggtggggaaagtcggaaaagtaacaaacccccaaacccagaaactgctgctggagttgtgccctgcacatgcaaagacgtggaccttgggtgttgctttaagggtttaccgcctgatctcgttaccccactaactgtgattgttactccatttgcatctcgtctgctgatttgccactgcctgccccctcttcctcaagaagaggccttcctggtgccaggctcaaaacccgaagctacagtttgccttatttttcactgtttggcttgatctgtaccatctctcccagccctaatgacagagaccttgccttaaagactttgcggttctgtaattgcagacttctccagcactcagaatcactttcaattccatagagttgttcctctccacacacacccctcccccggtgcacagaagggctgccgacacccaccagactcatacagagaaggtcacaagtactaaatgaaaggctcccagcccaccttgtctcagcccctgtcctgtgaagggactccttcctcacacagtttctgtgacttccttttgttcttaaacacatggaagttacttggcagggagacatttctagaataatttttgtgatcacgatacacccttcattctccgtttgctggtatgtttttagttggtttggttttagttttccacatacagtctagctcgtggccagccttgagctccgaggagatcctccatcttctcctctcaaatacccagggcccagacatttgcctctgcacctgctgggctttttctctcatttttccttaaccagaaaaacaaacgggtaatttcatcgactgcctatgtaggtttggtgtggttttgaaaaccttgtttccatggggccctttgggggagtcagggggcccctgcacgagcccatgcttcaatgccgagagattggctgctggaaccccctgaagcatgatttcatgttttagagatgagcttctgtggtacggtcatcatggtctccaacatcaacagacaaggtagaaggatgtcaacgacaaagataagttcagggctctttctagggtcaccactctgtcctgtgtaacactgacacagtctggccagcctttcaggaggtcctggctccctcctaaacccatgtaggwcttgctgcaagtaacagtgtatgaagtcaacctgacctggaacagaagtctgcatattggttaagatacagcctatgtatcttcatacaacgatccagaagtactagacatcatttttgcagtgaattatgggtaccatacatgactttgatgatatctgcttttaaacaaataaaatctttgatcactca

SEQ ID NO: 4 - DNA, 1663 bp, Rattus norvegicus
cctgggggtcgtgaagttccgagcggacgggtccacggaggttcatctggagaggtgggtcccctgcgaggtgaaaggcgccgctgcgacacacccccaccccccgcggtgcagtggttcagcccaataacttttcattcataaaaaagaccagactctgcgaggcgcgagtgagtcagaaccgcagccgccgacgcggaccctaccgaacatccagcccagggcatgtaccgagacttcggggaaccgggaccgagttccggggctggcagcgcgtacggtcgccccgcgcagccccagcaagcgcagacacagacagtccagcagcagaagttccaccttgtgccaagcatcaacgctgtcagtggcagccaggaattgcagtggatggtgcagcctcatttcctgggacccagcggctatccccgacctctgacctatccccagtacagtccccctcagccccgaccaggagtcatacgagccctagggccacctccaggggtgcgacgcaggccctgtgagcagatcagcccggaggaggaagagcgccgcagggtgagacgcgagcggaacaagctagcagctgctaagtgcagaaaccgaagaaaggaattgacagacttcctgcaggcggagaccgacaagttggaggatgagaaatcggggctgcagcgtgagatcgaagagctgcagaagcagaaggaacgccttgagctggtgctggaagcccatcgccccatctgcaaaatcccagaagaagacaagaaggacacaggtggtaccagcagcacgagtggggcgggtagcccaccgggcccctgccgcccagtgccttgtatctccctttctccaggacccgtacttgaaccggaagcactgcatacccccacgctcatgaccacaccctctctgactccttttactccgagtctggttttcacctatcctagcacaccagagccttgttcctcagcccatcgaaagagtagcagcagcagtggtgacccctcctccgaccccctaggttctcccacactcctggctttgtgaggcacccagccacaccccttgcaggtgctacccgttgtcatctcctttccctgttcatccagcaggcctggaccatacccatgccccaaaccagcaggtcttttatctctttcaagtagaacaaacatgttatgctttgatatagagccagcttgggggtccccaaagctgctcactgtttctctagagctggcctatcataatttgcatacagagaaaatatgtccctctgccagagtaagcctggcagctctgactttgtagatccccagtggtcctttgatgccttgcagaccactttcccacaccatgtcactttcttcatgttatccagcctactctaagcctagatagaaggtgccctttaactagcctagaacactaactcacacagcaccaacagccagcagcaccaggacaccctgtaggctcctcctgatcaggaggcaccaacagcttctgtgatgagctgagctgtactccctagctctgagaagcttttagctctggggtatccaagcctccacagcaagggcagctgctatttattttcctaaagagactatttttatacaaaccttccaaaatggaataaaaggcttgaagctct

SEQ ID NO: 5 - DNA, 1727 bp, Rattus norvegicus
  misc_feature (1628)..(1628): n is a, c, g, or t
cctcgtccttcttgctgtggcacttcagacctccgatgcaggtccctatggtgccaatgtggaagacagtatctgctgccaggactacatccgtcaccctctgccaccacgtttcgtgaaggagttctactggacctcaaagtcctgccgcaagcctggcgtcgttttgataaccatcaagaaccgagatatctgtgctgaccccaggatgctctgggtgaagaagatactccacaagttggcctagggagaagggcctgatgaccacgggtctggtgtctccacaaggctcagcaaaccctatccttctgccatccagcaagagccttgccaacaactccacctttgctcacctccatcccctgggttgtcactctgtgaagcctcgggtccctgtacttcctgtccgtcccctccagctcattctcttccaacgtggcagccgggaagcacttctggctagccttacccaatactactcccactgctttaaatgagacaggtccttgttttggtgcctttggatcctatgatgccttcccagtctccagccttggcccccttctcttcttacatgtagggaacaccaatatctttcaagtatgtgctacccaattcctcttcctcggaggctgctgggaccggaatataccctgctgcaggccctctccagcaccactcacctcccaggcttccatccgtcccagtcccaagccccatgcttcagacttccttggcccccacctacactcagaattctgggagtctcagactggtccactaggccccaagggaggaggtccccacaagagctcctcctgtttctcggtctccagcacaggagtgggctcagaatcagatttctgaaaggagctgcaaattctaccagtaaaattaatctttccccccatccctgatgtaggcaaaagacagtctcctctcaacacggagactcaggttgttagaaaggggctatgtaggtcaactgtgacctaaattcaccaagaatcaacttccatccatccctcttcaaccgaatgttaggtcttttactttttctacttctgactccatgcctgtgtagttgataatcaaagtcatgctatggtgacagtgatcgccatagcttggtattataagctatagttatatttatataggaaagaggataaatatatgtgaggccaaatagacgaactggagagtcttacgacctgcgggcaggaaggaccatacaaagcccttcagaaagcagctggttgtgggagctacaggcacagtggcatagttaaattaaagaacttagttgaaattattcttgagtgggctggggccaagtcaggaatttaaaaatcaattttggtttccctggagacaacagtgccccagggtggggaaataaatttgcttgctcctttggagggaacagtggcctggcttagctgagtgaatggattgcgtgagcctgaggacattgcttttcccctttgagcctcagctgacagaatatgggaccacaaagggttttccccaacccacagggactgacagtgccagccacagctgtgttcagggcttatgtccttccagaaggagcacccgatgaccagggccaccattagtggtantttgcccactgccatgcatgtcctgagggtcacctcctttcctaattttgggnaaataaatgcttgtcaatagtgcaaaaaaaaaaaaaaaaaaaaaaa

SEQ ID NO: 6 - DNA, 3259 bp, Rattus norvegicus
cagctccggcgggcagcaggcgctggagcgcatcgcagttcagctcagcgcagcaccatcggtctgcggagcggactgagctagaagcggagcgctgacgccggaggcgtgcaatgaggagggcaggtgctgcctgcagcgccatggaccggctgcgcctgctgctgctgctgattctaggggtgtcctctggaggtgccaaggagacatgttccacaggcctgtacacccacagcggagagtgctgcaaagcctgcaacttgggcgaaggcgtggcccagccctgcggagccaaccagaccgtgtgtgaaccctgcctggacaatgttacattctccgatgtggtgagcgccactgagccgtgcaagccgtgcaccgagtgcctgggcctgcagagcatgtccgctccctgtgtggaggcagacgatgcagtgtgcagatgtgcctatggctactaccaggacgaggagactggccactgtgaggcttgcagcgtgtgcgaggtgggctcgggactcgtgttctcctgccaggacaaacagaacacagtgtgtgaagagtgcccagagggcacatactcagacgaagccaaccacgtggacccgtgcctaccctgcacggtgtgcgaggacactgagcgccagttacgcgagtgcacgccctgggctgatgctgaatgcgaagagatccctggtcgatggatcccaaggtctacgcccccggagggctccgacagcacagcgcccagcacccaggagcctgaggttcctccagagcaagaccttgtacccagtacagtggcggatatggtgaccactgtgatgggcagctcccagcctgtagtgacccgcggcaccaccgacaacctcattcctgtctattgctccatcttggctgctgtggtcgtgggccttgtggcctatattgctttcaagaggtggaacagctgcaaacaaaataaacaaggcgccaacagccgccccgtgaaccagacgcccccaccggagggagagaaactgcacagcgacagtggcatctctgtggacagccagagcctgcacgaccagcagacccatacgcagactgcctcaggccaggccctcaagggtgatggcaacctctacagtagcctgcccctgaccaagcgtgaggaggtagagaaactgctcaacggggatacctggcgacatctggcaggcgagctgggttaccagcctgaacatatagactcctttacccacgaggcctgcccagtgcgagccctgctggccagctggggtgcccaggacagtgcaacgcttgatgcccttttagccgccctgcgacgcatccagagagctgacattgtggagagtctatgcagcgagtccactgccacatccccagtgtgaactcacagactgggagcccctgtcctgtcccacattccgacgactgatgttctagccagcccccacagagctgccccctctccctcggggatggcccaacggtcagaacggagcatctctgtgcagggcctctgtgttcccactcctgactccgttgctgctcccgagggggcccttgcttctgaccaccctctcctcagcaagagagagagagaggaccacccgagcctgacttgctccatttccatctcaggcctttccttcctttctacacattagctgtgtcagatctgggggtttgacactaggagaagggagcgggggcacccctaagactcaggaggtactgaagaaccagagccatggactccacactgtgaaccggagaacaaggggcggggcattgtggtaggctagaccttccttagcccctcccttctcccctctggccaaagaagaggattacggacctatctgagctgaaagcaggtttggaacccagcccacacttctctctcacacacaggatggtaaaacccagagaaaggcagggactgacctaggccacccaaccacaggaagaacaaatgaaggctgatacactccgtttctgaatgagggcgtcaagtgtgcttgttgacagggatggcgtgactttcagggaaatatctggaagccatgtctgccccgccctcaaccacttccaggcccctacccaacccttgtgcagatgaactgtttgttcaagggctggtccattggtctattctgatggagtcaagctaagggctcaggcttatccataaggcatttgtggagagatgaatctgttagtgcgctcattcttggcataagcctgaagccaacacggcccttaatgtcagccctcggggtcaggaaccaaggactcccaccccacaatccaacactatactacattacacacacacacacacacacacacacacacacacacacacacacacagatatcttgcttttctccccatggctcttttggggctgagactagatcctgctgggagtcactgccagtgagagatccggaggggacagagctgagcttcatggggctgtcttcctcgcccccgggtctggcaggccaagaatgactgcatctgagctggtgtctgtcttccaatggcctgtgcgtggaggaaatgctcccactcctccccttcttgaagctgcccccagaagactacagtgcaaaagagcagactggtgtgagaacacaagaaaaagcagatgctggccctgcagtctgtggcagctttctcctcagcttcaaggcccctgcaaaggacggatttcctgagcacggccaggaaggggcaagagggttcggttcagtggcgctttctcccggctccttggcctgttctgttttgcttgctgttggaatgagtgggcaccccctctatttagcatgaaggagccccaggcagggtatgcacagactgaccaccatccctccccacccagggtccacccaacccggtgaagagaccaggagcattgtacgcatacgcgggtggtatttttatggaccccaatctgcaattcccagacacctgggaagtgggacattctttgtgtatttattttcctccccaggagctggggagtggtggggggctgcaggtacggtttagcatgtgtttggttctgggggtctctccagccttgttttgggccaagttggaacctctggccctccagctggtgactatgaactccagaccccttcgtgctccccgacgccttccccttgcatcctgtgtaaccatttcgttgggccctcccaaaacctacacataaaacatacaggaggaccattaaattggc

SEQ ID NO: 7 - DNA, 5810 bp, Rattus norvegicus
cctgcacctccgagaatgactgtcgaatcaaccaacgagaggtcttctgggctggtaactgctcagaggccgcatgcggggctgccgactgtgagcagtgcacacgggagggaaagtgcatgtggactcggcaattcaagaggactggggagactcggcgtatcctgtcagtgcagcccacctacgactggacttgcttcagccattctctgctgaatgtgtctcctatgccggtggagtcctcacccccactgccctgtcccaccccctgtcacctcctccccaattgcacatcctgcctggcctccaagggtgcagatggaggctggcagcactgtgtgtggagcagcagcctccagcagtgtctgagcccctcctacctgcccttacgctgtatggctggaggctgtgggcggctgcttcgggggccagagagctgctccctgggctgtgctcaggctacgcagtgcgccctgtgtctgcgaagaccccactgtggctggtgtgcctgggggggacaggatggaggtggccactgcatggaaggtggtctcagtggcccccgagatgggctcacgtgtggacgtcctggggcatcctgggccttcctgtcctgcccccctgaggacgagtgtgcaaacgggcaccatgactgcaatgagactcagaactgccacgaccagccccacggctacgagtgcagctgcaagacaggctacaccatggacaatgtgactggcgtctgccgccctgtgtgtgcccagggctgtgtgaacggctcctgtgtggagcctgatcactgccggtgtcactttggctttgttggccgcaattgctccacagagtgtcgttgcaatcgccacagtgagtgtgctggcgttggagcacgggatcactgcctgctgtgccggaaccacaccaagggtagccactgtgagcagtgtctcccactgtttgtgggttcggctttgggtggcgggacctgccggccatgccatgccttctgtcgagggaacagccatgtgtgtgtctccaggaaagagctagaaatggccagaagggagccagagaagtactcactggatccagaggagattgaagcctgggtggcagaagggcctagtgaagatgaagctgtgtgtgtgaactgccagaacaacagctacggagacagatgtgagagctgcctgcacggctacttcctcctggacgggaaatgcaccaagtgccagtgcaatggccacgctgacacctgtaacgagcaggacggaacaggatgcccatgtcagaacaacacagagacaggcgtctgccagggcagctctcctagtgaccgccgagactgctacaagtaccagtgtgctaagtgccgggagtcattccacgggagcccactgggtggccagcagtgttaccgactcatctctgtggagcaggagtgctgcctggaccccacttcccagaccaactgcttccatgagccgaagcgccgggccttgggtcctggccgaactgtgctgtttggcgtccaacccaagttcaccaatgtggacatccgcctgacactagacgtgaccttcggtgctgtggacctctatgtctccacttcctatgacacttttgtggtccgagtggcccctgacacaggtgtccacactgtgcacatccagccacccccacctcccccacctcctccgcctcctgccgatggtgtacctcgtgtggcttcggacctaggaggactggggaccggaagtggatcaggttctccagtggagccacgagtccgcgaggtgtggccacggggcctgattacctatgtgacagtgacggaaccctcggcagtgctggtggtccgcagtgtgcgggacaggctggtgatcacctatccccatgagcaccacgccctcaagtccagccgcttctacctgctgctgctgggggtaggggatcccaacgggcctggggccaacggctcggcggactcacagggcctgctcttcttccggcaagaccaggcccacattgacctgttcgtcttcttctctgtcttcttttcctgcttcttcctcttcctctcactctgtgtgctcctctggaaggccaagcaggctctggaccagcggcaagagcagcgccggcacctgcaggagatgaccaagatggctagccgcccctttgccaaggtcacagtctgcttccctccagaccctgcgggcccagcacctgcctggaagccagctggcctcccgcctcctgccttccgccgctccgagccatttcttgcacctctactgctgacaggagctggggggccctggggacccatgggaggagggtgctgcccaccagctctccctgccaccacagctggcctacgagctgggcccattacccttgaacccacagaagatggcatggctggtgtggccactctgctgctccagctacctggggggcctcatgctcccaatggggcctgccttgggtcagcccttgtcactctgcgtcataggctgcatgagtactgtgggggaagcgggggtgctgggggcagtggacatgggggtggtggaggccggaagggactgttaagtcaggacaatctcactagcatgtccctttgacctgtcaaggttctcttccgcagttaccacgttgcctgatggggccactctgggctagagatcccctggacagtcctcagaaaccactggctccctttctccagggtctcccctttgttctgcatctagcaactgtttattgagctactactgtgtgcctcgtacagttgtaggcacagagcaaggcaggaggtgatagcccccacccccatctgctaggacttgagttcttgtgggaggagacacgcacatgtaacacagtgaggcatccaggtaaagtggatgcggcacttggagaaactgggctgagaaaggtgacagaatgagggaagacctttggaacgcaaatttcaagagttgaggaaactgtaaggccgaaggcctgaggctggaagcggtttgttttattacaagaaccaggaacccggccatcaagtgtagactgagctgggttttattctcagggcagggaaagtggttgggccgttctgtggaggtaaatggagagtctcagctctgggggggaagagtgggctgatgggcacagagaggctgctgcaggtgggcaactgggcagaaggagggtgagtggcaggatgtgacagtggtgcagacattagggtaaaggagaggacatatccagaatggccactgggtggacagtgaggtcattactaagctgggacaggctggggcttgtgggctggagccgggtgtgactggattcatacggcaagagcttgagtgcatcaacaggagggatctagtgcactgcggtgagctggcaaaggcaggcaggtacggggaggacaggacagtgcaactacccgctgtaggcacagtgagctgctgcctaccctgggctggaccagggtctctagctgaacctggagatgtaggcttatttcaagtgagcctcagaggcaaatccttcacctgagatgcctcacaccctgctctggcagtgtggggtgggggatcctggaggggtcatcatttggctctttttgagtccttgggactctcctcagaggccaccccaacctgggtctcccttttggacttaggagctctctcttcccccagtcctcggcagtgagcttgggactcctgctctccccaggacacagcttcaagcctcggcggctgctgctgctgctgctgctgctgctgctgctgctgctgctttggtcctgggatgggggttgggcctagcctctttcccagtctgataggtcctgataatgtgcatgtgtcagcagggctcacatttctttttttttttttggttcttttttttggagctggggaccgaacccagggccttgcgcttcctaggtaagcgctctaccactgagctaaatccccagccccagggctcacatttctacaacaggagggcaactgaggcagcaataaagtgtggggaagactcgggagggcggcttgactgccttctgtgtgccggggatgagggatgctctgggtgagtggaacatgtgtcctggtcgcacctccaagctgtgtatcctgagcctagctctgccccttctcagcaatgcagggtgctgcaacggggtctcctgctccccggaagagggatggcaggaacaatgggcagggacatctttgaccgtttctttgtccctgccacagttcggctcagggagaatcagacagtctgtgtcacagggattgtccttagataagaaaagcccaggtacgtacggaacacagaaaccattgtttgaacgggaggaattgagcacagagaactggtagtgctggtgagggcagcagctgcaggacagtggcagctgggtgtggacaagcattctcagaagtaagctcgacatcaaagctgagaaactattacccccccaccccgccttcccatcctagccctcggccccggacaccaaaatctggacgcaagggcagaggtggacctgacatctcggcctcagctccacacggttctttgctgcagaggctgaacagtgcattttgtctgtatgcttggccccaatgccagtcccttgcttcagtgcatggccgtgtggcgcatgagtgacagtacgttcaggcggtactctggccccatgtgagttttgctttcatgagctgacagtgtctcctgccctgtgagatgtgaacataaacttggcttttaaaaaaagtaattttgtgtaagtatgtgtgcatgtgggtggaggtgccctaaggaggccaggagaaggcatcagatcctctcaaactagagttgcaggcagtcatgagctgccatgtgggtgccgggaaccaaagtgctccttcccaagggcagccagagctcttaaccactgagccatctctcactgtagtcggtttacatattaagagctgctaccatcagaggtgaacacaagcttggaaaggtttattcgttccccaagattgcacggaaatggtaaggtaaagactgggtcccaggagttctgagacccagcatgttccagttaattccttctgcagtacgtgtcgccgggcgaaacgtcactgagttgaagaggtggtaatgaacttagtatgatgagctagctcatgagggaccaccgttggtggccttcccccaaaggcccatcttgtctggaagcagatgggaggacaggtgtgggggatccaactccctttgggaactccatgtatgtggtgtgtgcaggcacaagacacacacacacaattaaaattcctttttaaagtatctgtagatactgagcgagctccggagagcattgtttgaagagtttaagtctttctttcttaatattttacaagttttctcttatttgaagcagggtctcaatatgcggcccaggctgccctcattccatgcagcctgggcttctcttagacttcctgcctcagcatctgtagcgtggggattgcagcaggcttgggcgtctatccccagactgtctggttttgcttattttcaggatgtgggcaggacttcagataggtcatctgcagcctcaggctaatggttctactttgtgaggtgctcccaggaagtcctcacaggcccacccctagcaaacccgtcttgtctgatacacctagatgtctctctcagcaaacctcccaatgcacccagagtccttccccaagacaataaaaacttgtaggtggggttggttttcg

SEQ ID NO: 8 - DNA, 3565 bp, Rattus norvegicus
cacgagctcgttcccctcgcagcctcagctaagacccggagaggtggaatttcgatttgaaactccctagccagggccggggctgggcatgctcagtgcctcggtgtatcgggctgctgctagctgcacaggcagcagaggcgctgcctggcttggaagctggcacaagagtccaccccactgtcaccaccgactgcgggcttcaggaaggccaaacactcacctcctctgagcgctttacacccaccaacctcgagtccccatctcccgcagcatcctactctcccgtctccccatcccttagggagacgaagccagtgctcggttctgtaggcacgccacagactgctctgtgaaccccagcggaggaccctgtcatcggatgcctcgggatcaaaggactcttggcagtgtttctctcaaatagcactttgcctggagtgggattgcggtggagtttctcccccgcccctcaatgcacacactatgaggagagcggtcggtttccctgcactgtgcctgcttcttaatcttcacgctgcagggtgtttttccaggaacaatgaccactttttggctattcgtcaaaagaagagttggaaacccatgttcatttatgaccattcccaagacattaagaagagcctggatatcgctcaagaggcatataaacacaactaccccgccccctcagaagttcaaataagcaaacgtcaccagatcgttgattcagcatttcctagacctgcatatgacccatctcttaatctgttggctgcatctggtcaagacctcgaaatagaaaatctcccaattccagcagcaaatgtgattgtggtgacactgcaaatggatatagacaagctgaacataaccttgcttcggatcttccgccaaggagtagctgcagccctgggactcttacctcagcaagtacacatcaaccgcctcattgaaaagaagagccagattgagttgtttgtgtctcccggaaaccggaaaccaggagagccgcaggccctgcaggctgaggaagtactgcgttcccttaatgtggatgttttgcgtcagagtttaccacagtttggaagtatagacgtctcccctgagaaaaatgttttacaagggcagcatgaagcggacaagatctggagcaaagaaggcttttacgccgtcgtcatcttcctcagcatctttatcattatagtaacctgtttgatgatcatttataggttaaaagaaaggcttcagctttccttccgacaagataaagagaaaaaccaggagatccacctatcgcccattgcactccagcaagcacaatcggaggccaaggcagcccacagcatggtccagcccgaccaggcgccaaaggtgctgaatgttgttgtggaccctcaaggccaatgcactcctgagattcgaaacaccgcctccacttctgtctgcccttctcccttcaggatgaagcccataggactccaggagcgacgaggttccaatgtatctcttacactggacatgagtagcctgggcaacgtggaaccctttgtggccgtctccaccccccgggagaaggtagccatggagtacctgcagtcagccagccgagttctcacaagtccacagctgagggacgtcgtggcaagttcccacctgcttcaaagtgaattcatggaaataccaatgaactttgtggatcccaaagaaattgatattccacgtcatggaactaaaaatcgttataagaccattttgccaaatcccctcagcagggtgtgcttaagaccaaaaaatataactgacccattgagtacttacatcaatgctaattacattcggggctacagtggcaaggagaaagccttcattgccacacagggccccatgatcaacactgtgaatgacttctggcagatggtttggcaagaagacagtcctgtgattgttatgatcacgaaactcaaagagaaaaatgagaaatgtgtgctctactggccagaaaagagagggatttacggcaaggttgaggttctggtcatcggtgtcaatgaatgtgataactacaccatccgcaacctcgtcttgaagcgaggaagtcacacccaacatgtgaagcattactggtacacttcatggcccgatcacaagactccagacagtgcccagccccttctgcagctcatgctggacgtagaggaagacagactggcctctgaaggccgagggcctgtggttgtccactgcagtgcagggattgggagaaccggatgtttcatcgctacatccattggctgtcaacagttgaaagaagaaggagtcgtagatgcactaagtatcgtctgccagcttcgtgtagacaggggtggaatggtccaaaccagtgagcagtatgaatttgtgcaccacgctctgtgcctgttcgagagcagactttcgccagaaacggtccagtgactctgaagatctaccagagcgtcaatctatcacgggtgattcgtcaagttacctactgagggctccgagaaggagctccccacgatggatgaggaggctctaaagccagcctgaggcgtggattgtggaagctatggtaacttgaaagattgccacctttgtgtataggactgtgttctaggcatccccctaggtactccgaaggacctgtgctgatggaggtgtgacagtcctcatgcagcctgaggaggattctgttttgtctgtgtcgactctcacacagaacctgtatgtgtaatattcagagtctgtgcttatggtgactggaggagctgcggaagatatcgttatcctgtgttagatgctttaatgttcacaaagcctgtcttgtgactggactgtcagctgttcaactgttcctgtattaagtgctattacctatctcagttaccagagtcttgctgctaaagttgcaagtgaccgacaatggatttttaaccatgacgttttttgtttttgaaaaaaaaaaatcaaaggcagtaactattttatatggagatgtgtcttgattatctctaataagtgtgtatttatagtatctcctatggatcaattacagagcacaatgattgtcactggatatatatgtatatgtacattacaggtatatgtacgcatatatattctattattaagcatagaggtagctcctgttttacaactctaactactatatttctacattgtgacttgttttactttaaaaggggataaacaaatttatagtaactataaagtatcaataattttaaatactcatctctcttttactaagaagggatttttgaatatagtgtctacttggtttctctctcccagtaagaagcaggtgcctttggggatgctccaacctgtttcatccccgaaagtttttctgaggacaccgtcatgcacgcatgaccttaaatttctaaaaggttttcttcctcaagagaccaatgaaggcaagataaccgcactcagcaaacatgagatgttctgctcagatgtgaattttcaatgcagcaaagttgactttcgtactcccaataaatgactcttaacagcc

SEQ ID NO: 9 - DNA, 2704 bp, Rattus norvegicus
cgctgacggcacgccgctccggcgtcccctgcatccctcgctgcggcctgcggggtcctgctgggaaccccggcctctcgaggaggcctggccccgagagccgccaagtctcgccgcgtctcgcgagagtccaagttgagaacatggcgacgtctaatctgttaaagaataaaggttctctccagtttgaagacaagtgggatttcatgcgtccaattgttttgaagcttttacgccaggaatctgtaacaaagcagcagtggtttgatctgttttcggatgtacatgctgtctgtctctgggatgataaaggctcatcaaaaattcatcaggctttaaaagaagatatccttgaatttattaagcaagcacaggcacgcgtactgagccatcaagatgacacagctttgctaaaggcatatattgttgaatggcgaaaattcttcacccagtgcgatattttaccaaaacctttttgtcaactagaggtgactttattgggtaaacaaagcagcaataaaaaatcaaatatggaagacagtattgttcgaaagctcatgcttgatacgtggaatgagtcgattttttcgaatataaagaacagactccaggatagtgcaatgaagctggtgcatgctgagagattaggggaagcttttgattcccagctggtcataggagtgcgagagtcctatgttaatctttgttctaatcctgaggacaagcttcagatctatagggataattttgagaaggcatatttggactcaacagagagattttatagaactcaggcaccctcatatttacagcaaaatggtgtacagaattacatgaaatatgcagatgctaaattaaaagaagaagaaaaaagagcactccgatacttagaaacaagacgagaatgtaactctgttgaagcactcatggaatgctgtgtgaacgcactggtgacctcatttaaagagactattttagcagaatgtcaaggcatgatcaagcgaaatgaaactgaaaagttacatttgatgttttcactgatggacaaagttcctggtgggatcgagcccatgttgaaggatctggaggagcacatcataagtgctggtctagcagacatggtggcagctgctgaaaccatcaccactgactctgagaagtatgtggaacaattacttacactgttcaatagattcagtaaactggtcaaagaagcttttcaggatgatcctcgtttccttactgcaagagataaggcgtataaagcagttgttaatgatgctaccatatttaaacttgaattgcctttgaagcaaaaaggagtggggttgaaaactcagcctgagtcaaagtgccctgagttgcttgctaattactgtgacatgttattaaggaaaacaccattaagcaaaaaactaacctctgaagagattgaagcaaagcttaaagaagtgctcttggtacttaaatatgtacaaaacaaagatgtttttatgaggtatcacaaagctcatcttacccgacgtctcatattggacatctctgctgatagcgagattgaagaaaacatggtggagtggctaagagaagttggtatgccagcagattatgtgaacaaacttgctagaatgtttcaggacataaaagtatctgaagacttgaaccaagcttttaaagaaatgcataaaaataataagttggcattaccagctgattcagttaatataaagattttgaatgctggtgcttggtctagaagctctgagaaagtctttgtctcacttcctactgaactggaggatttgatacctgaagtagaagaattttacaaaaaaaatcatagtggtagaaaattacattggcaccatctcatgtcaaatggaattataacatttaaaaacgaagtaggtcagtacgatttggaagtaaccacgtttcagttggctgtgttgtttgcatggaaccaaagacctagagagaaaatcagctttgagaatctaaaacttgcaacggaactccctgatgctgaacttagaaggactttatggtctttagtagcttttcccaagctcaaacggcaagttttattgtacgaccctcaagtcaattcacccaaagattttacagaaggcaccctcttctcagtcaaccaggactttagtttaataaaaaatgcaaaagtacagaaaaggggaaaaatcaatttgattggacgcttgcagctcactacagaacggatgagagaagaagaaaatgaaggaatagttcaactaagaatattaagaacccaggaagccatcatacaaataatgaaaatgaggaagaaaattagcaatgctcagctgcagactgaattagtagaaattttgaaaaacatgttcttaccacagaagaaaatgataaaagagcaaatcgaatggctgatcgagcacaagtacatcaggagggatgaggccgacatcaacaccttcatatacatggcgtagctgaatccagcctgccgcagctacacatctggagacctgggcagaaggttgtcccaggctgagctggaggaaggtttatttggactttgattacataaatattaaactctgccttaccttacaaagcgactatattttgccagtcacattagtcagcatgatggcaatcccttcatgttgcacactctttaacagcatgctgttttgtggagaaaatt

SEQ ID NO: 10 - DNA, 2301 bp, Rattus norvegicus
ccccgtgtccctcgcgcaggcgggcggccccggagacatagtgcccgcaaaggcggtgacggcggtgcgcccactcatcatgagcacaagctttgcccgaaaaagccacacattcctgcccaagatcttcttcagaaaaatgtcatcctcaggagctaaggacaagccagagctgcagttccctttccttcaagatgaggacacggtggctacgctgcacgagtgcaagacgctgttcatcctgcgtggcctgccgggcagcggcaagtccactctggctcggctcatcgtggagaagtaccacaacggcaccaagatggtgtctgctgatgcttacaagatcattcctggctctcgggcagacttctccgaggagtacaagcgtctggacgaggacctggctggctactgccgccgggacatcagggttcttgtgctcgatgacaccaaccatgagcgggaacggctggaccagctttttgaaatggcagaccagtaccagtaccaggtggtgctggtggagcccaagacggcgtggcgactagactgtgcccagctcaaggagaagaaccaatggcagctgtcgctcgatgacctgaagaagctgaagcccgggctggagaaggacttcctgccactttactttggctggttcctgaccaaaaagagttccgagaccctccgaaaagccggccaggtctttctggaggagctgggaaatcacaaggctttcaagaaagagcttcgacacttcatttctggggatgaacccaaggagaagcttgacctggtcagctactttggcaagagacctccaggcgtgctgcactgtacaaccaaattctgtgactacgggaaggccaccggggcagaagaatatgcccaacaggatgtggtgaggagatcttacggcaaggccttcaaactgtccatctctgccctctttgtgacacccaagacagctggggcccaggtggtgcttaatgagcaggagctgcagttgtggccaagtgacctggacaagccctcatcctccgagagcctgcccccagggagccgagctcacgtcaccctaggctgtgcagccgacgtgcagccagtgcagacaggccttgacctcttagagattttacagcaggtgaaggggggcagccaaggtgaggaggtgggtgagctcccccggggcaagctctattccttgggcaaagggcgttggatgctgagcttggcgaagaagatggaagtcaaggccatcttcacggggtactatgggaagggcaaacctgtacccgtacacggcagccggaaggggggtgccatgcagatctgcaccatcatctgagggttcacgccactgtgccaccgctgtggacacagcaacatgccttccacttgatttttaagattttttttttttactcaaagctaatctacctataactttttagaagtctgtaaaataaccatcagttccttcacccaccctcttccccttaacactcagctcaacacaggcaggtgggcagaggaagacaccattcaggaacctggaccagaggtggtaattaaagggctgggtcattggcctggagcggccacctagatgcacccaggaccttgttaaccctcccttagccgctgcccatgcagagctgggcacacacacctcagagccaccacacatcatgcaagggacaaatgctactgccacaggaaaacagtggtagctttgagaggggacagacccctgggtgccaatcttaatcactgtagccccaaggaaggcttgggctacttactgggctgtaaaaggggcttacctctcaccgttggtcattcccaactccctctctccttcactggcactaggttaatggtgtgaccactaggggagaaaatggcattaccggaagccacatctctggttccggtctggctatctagccttaacagctgtctctgctaaggtcagagagatgctttaagaaactccagctttgctttctgcccagcccatcttgcctccgtggtgcctctgggcctcctctaaaggatggtggtgggtaccaagagccaacggagggggccctgagccagcaagagtaagccccatctgctctggggaagtctgaacacgcgcgtgtgctgtggttcctggagtacctcactctgctcagtaacaggaccttgctaattgggttgtcacccaagcatctgggatttatgttcctaccctcaccctgtgttgcccacctcagcaacaactaagactgatactgaaataaatcgtgttaatcccaa

SEQ ID NO: 11 - DNA, 771 bp, Rattus norvegicus
atgcagaggaccaaggaagcaatgaaggcatcggatggcagcctcctgggagaccctgggcgcataccgctgagcaagagggaaggcatcaagtggcagaggccacgtttcacccgccaggccctgatgcgttgctgcttaatcaagtggatcctgtccagtgctgccccacaaggctcagacagcagtgacagtgaactggagttgtccacggtgcgccatcagccagagggcttggaccagctacaggcccaaaccaagttcaccaagaaggagctgcagtccctgtaccgaggcttcaagaacgaatgtcccacgggcctcgtggatgaagataccttcaaactcatttattcccagttcttcccccagggagatgccaccacctatgcacacttcctcttcaatgccttcgatgctgatgggaacggggccatccactttgaggactttgtggttgggctctccatcctgcttcgagggaccgtccatgagaagctcaagtgggccttcaatctctacgacatcaacaaggacggttacatcaccaaagaggagatgctggccatcatgaagtccatctacgacatgatgggccgccacacctaccctatcctgcgagaggacgcacctctggagcatgtggagaggttcttccagaaaatggacaggaaccaggatggagtagtgactattgatgaatttctggagacttgtcagaaggacgagaacatcatgagctccatgcagctgtttgagaatgtcatctag

SEQ ID NO: 12 - DNA, 794 bp, Rattus norvegicus
agcacccacctagacttgggtatcagctacaccaacaggtctcccccgacaggagccgaatcccggagttatgtctgaagagcaagaacccaagacttctgaagcagaatacggtacaatggactttcctgaattcgaaaatgaagaagaatggcttttcaaagttctgggaatcaagcctaggccttcctctgatctggatgacgctgataagcaggaagatgagccactgggccacaccgaatttcttcgcctgcaagatattcttcaagaggacaaagtcagcagtaccaatgatagcgacacttgccaagctgggtataccgaagaaaatgatgaggccagccacagtgacagcgacattgacgataatgtgaacgtcatcattggtgacattaaagcaaactcctccatgtatatggagatgttcactaatatgaactcacaagctgaccaagacctgaaactaactgaatcagacaatgccatgtacccaactgattaaacaagcaaggatctgaaacgtgtcaccatcatcatccttattggcctcatgcattacaaattaaatggtgatgggaaataaaaccttgtttttaaagaaaatgacatcatctttacacaccaacctaaaagcaaatttataccaaactgctacaacaaatggagagtacttaatttgagacctgactgttttgaacgttggaaatcctaacattctaaggtcatctacaagccaactagtggaatttgttgcttttggaaggttgaataaaatctgcttgaaaccttta

SEQ ID NO: 13 - DNA, 1403 bp, Rattus norvegicus
gggcttgcggcttctaaaatgccggattacctaggtgcagaccagcggaaaaccaaagaggatgagaaggacgacaagcccatccgagctctggatgaaggggatatcgccttgttgaaaacttatggccagagtacttattcgaggcagatcaagcaggttgaagatgacattcagcaacttcttaaaaaaattaatgagctcactggtatcaaagagtccgacactgggctggcccctccagccctctgggatttggctgcagacaaacagacacttcagagtgaacagccgctacaggttgcaagatgtaccaagataatcaacgccgattcagaggacccgaaatacatcatcaacgtgaagcagtttgccaagttcgtggtggatctcagtgatcaggtggcacctactgacattgaggaagggatgagagtcggcgtggacagaaataaataccaaattcacattccactgcctcctaagattgacccaacagttaccatgatgcaggtggaggaaaaacctgatgtcacatacagtgacgttggtggctgtaaggaacagattgagaaattgcgggaagtggttgaaacccctctactccatccagagaggtttgttaaccttggaatcgagcctccgaagggtgtgttgttatttgggccaccaggcacaggcaagaccctctgtgccagggcagtggccaatcggactgatgcttgcttcatcccagttatcgggtctgagcttgtacagaagtacgtcggtgagggggctcgaatggttcgggagctttttgaaatggccaggacaaaaaaagcctgccttattttctttgatgaaattgatgccattggaggggctcggtttgacgatggcgctgggggtgacaatgaggtgcagaggaccatgttggagctcatcaatcagctggatggctttgatcctcggggcaacatcaaagtgctcatggccactaaccgacctgatactctggacccagcgctgatgaggccagggaggctggacagaaagattgagttcagcttacctgacttagagggtcgaacgcacatctttaagattcatgctcgctcaatgagtgtggaaagagacatccgatttgagctactagcccgcctgtgtccaaacagtactggagcggagattagaagcgtttgcacagaggctggtatgtttgcaatccgagcccggagaaagattgctacagagaaggacttcttagaagctgtaaataaggtcatcaagtcgtatgccaaatttagtgctactccccgctacatgacatacaactgagccacagaggctttgactgaaggaacttcctgtgcgttcataacttgttaccgcctcacaaaaataaaagacttaaaaactgt

SEQ ID NO: 14 - DNA, 3101 bp, Rattus norvegicus
tgtctacaccataattcctttgtctttgagccagctcacaaatgtcactgtggttctgagtgtgggggtcttggtgcagtccctcccctcccagtcccttccgtcgaggagcatggtgctagtgctgccacagcctggagacgcacacaaccccccaaaatctctccagacgaccgtcccacgatcacaggacagaaccctccaaatcgaaacggaggaaacggacagccattgaacatggacgaaggaatccctcatttgcaagagagacagttactggaacatagggattttataggactggactattcctctttgtatatgtgtaaacccaaaaggagcttgaagcgagacgacaccaaggatacctacaaattaccgcacagattaatagaaaagaagagacgagaccgaattaatgaatgtattgctcagctgaaagacttactgcctgaacatctgaaattgacaacactggggcatctggagaaagcagtagtcttggaattaactttgaagcacttaaaagctttaacagccttaacggagcagcagcatcagaagataattgctttacagaatggggagcgctctctgaaatcgccggtccaggccgacttggatgcgttccactcggggtttcaaacctgcgccaaagaagtcttgcaatacctcgcgcgctttgagagctggacgcccagggaaccgcgctgcgcacagctcgtcagccacctgcacgccgtggctacccagcttctgacgccacaggtgaccccaggcaggggccctgggcgcgcgccctgcagcgctggggctgcagccgcctccggttccgagcgcgtcgcccgctgcgtgccggtcatccagcggactcagcccggcacggagcccgagcacgacacggacaccgacagcggctatggaggcgaggcggagcagggccgcgccgccgtcaagcaggagccacccggggacccgtcgctgcgcccaagaggctgaagctggaggcgcgcggcgcgctcctgggcccggagcccgcgctgctcggctctctcgtggcgttgggcgggggtgcgcccttcgcgcagcccgccgccgcgcccttctgcctgcccttctacctgctgtcgccgtccgccgccgcctacgtacagccctggctagacaagagtggcctggacaagtatctgtaccccgcggcggccgcgcccttcccgctgctgtatcccggcatccccgcagcagccgccgctgccgccgccgccgctttcccttgcttgtcgtccgtgctatcgccacccccggagaaggcaggttcggccgctggtgccccattcctggcgcacgaggtggcgcccccggggtcgctgcgcccccagcacgcgcatagccgcacccacctgccgcacgccgtgaacccagagagctctcaggaagatgccacgcagccggccaaggacgccccctgaacccagcattccttccagaacagggcagggggctcctgaggagtcgccaggtttccaagttcaaacatcccctaaggcgtaccagggaggaagagtaagagatgctctgctcgacaggcttaggacaaaaacaggtgttttgtgtatgtttggagttcctgttttgcccctttctcacccttctgccaccccaccctctaccctttgacactcccttccccatccctgctgtcacagagcctccctgagaaatactggttatcttaaattaccctcccttacatttagttcacgtcctctgtttccaaacatagaccctggttcaggagtctgttgggtgggagagccacacggaaccagttagagtgcctggtatcagggctccttgacccaggcctggaacagtagctgtgtcccctgtctgtccccttaggaggtgacccataactgagggtctctgaaagttacattgacgtgtcagtattttgtattcttcagctttttggaaggtacctctttttcaaagaagtgaggatgccattgccctgttgtgaggtggctggagtggtgtctttataccttgcacctgttgggagaaactgagagttggggccatcttcaggcactgtgtcagtgtgggagctggaagagggagtttggagcccgtggcgcctttctcgcactttattgacaaattgacctcaacccctttgtcccatgtctcaactcacagatatatgtcataggttatatatttgtgtttctgatccctcgttattttatccatcatggtcccaaatttttgtaatgttactggggtttggggtggggtggggtgttaaagtgctctgggctggaaaaagacaagcccaaacctattgattgtcgaattcttagatgacagaagtggagagaggggcttgtggtcccttgtgatgggaagtgctgtgaacatgtagaaggccctgccagcctcgctctctcaagtctgtatgtatttttcgggagaccaaaccagacaccagataatcaggaagaaagctttttaaaataaggcaaaaaccgagaccttgtctagatatttttagtttgttgccaaggtagcactgagaaatctcacttgaatgttacataaggagtgattcacaatagtctagagtgaagaaagttatctgggtctgtgagtgttcgggtccgtttgctgctgctgttgctactgtttgcctcaaacgctgtgtttaaacaacgttaaacttcttagcctaccaaggcggccgtatgtacatagctgttaatacccccaactaatgtctgacatgctatttttgtagggagaagatacctgctagtgatattttgagttaaaatatcttttggggcggacttggtgaaatgtttgcactttggtcacaatgcttctactgcttggtgcaacgttacgctgtcttaaattattaaacaaataaaaaatactatctgcaagaaaaaccagctggtttagacaagtttagtatgtaaagataagctagaaactatctttatattctagtattttcagcactccatattacctaaatattgccacactattttgtgatttaaaagttcttactaaggaataaaatctttata

SEQ ID NO: 15 - DNA, 2006 bp, Rattus norvegicus
gccggcgccgcagctgccgggcctctcgagtcggtggacgcgccctcccgccggcctcgccgcccgcgcaaccgccgtccttcggcctcggctgcgtcgcgccatggcggccccgggcgcccggcggccgctgctcctgttgctgctggcaggccttgcacacagcgccccagcactgttcgaggtgaaagacaacaacggcacagcgtgtataatggccagcttctctgcctcctttctgaccacctatgatgctggacatgtttctaaggtctcgaatatgaccctgccagcctctgcagaagtcctgaagaatagcagctcttgtggtgaaaagaatgcttctgagcccaccctcgcaatcacctttggagaaggatatttactgaaactcaccttcacaaaaaacacaacacgttacagtgtccagcacatgtatttcacatataacctgtcagacacacaattctttcccaatgccagctccaaagggcccgacactgtggattccacaactgacatcaaggcagacatcaacaaaacataccgatgtgtcagcgacatcagggtctacatgaagaatgtgaccattgtgctctgggacgctactatccaggcctacctgccgagtagcaacttcagcaaggaagagacacgctgcccacaggatcaaccttccccaactactgggccacccagcccctcaccaccacttgtgcccacaaaccccagtgtgtccaagtacaatgtgactggtgacaatggaacctgcctgctggcctctatggcactgcaactcaacatcacctacatgaagaaggacaacacgactgtgaccagagcattcaacatcaacccaagtgacaaatatagtgggacttgcggtgcccagttggtgaccctgaaggtggggaacaagagcagagtcctggagctgcagtttgggatgaatgccacttctagcctgtttttcctgcaaggagttcagttgaacatgactcttcctgatgccatagagcccacgttcagcacctccaactattccctgaaagctcttcaggccagtgtcggcaactcatacaagtgcaacagtgaggagcacatctttgtcagcaaggcgctcgccctcaatgtcttcagcgtgcaagtccaggctttcagggtagaaagtgacaggtttgggtctgtggaagagtgtgtacaggacggtaacaacatgctgatccccattgctgtgggcggggccctggcagggctggtcctcatcgtcctcatcgcctacctcatcggcaggaagaggagtcacgcgggctatcagaccatctagcctggtgggcaggtgcgccacagacgcacacgggcctgttctcacatccccaagcttagataggtgtggaagggagggaggcacacttgtggcaaactgttttcaaatctgctttatcaaatgtgaagctcatcttgcaacatttactatgcacaaaggaataactattgaaatgacggtgttaattttgctaactgggttaaatattttgctaactggttaaatgttaatatgttaccaaagtagagctctaaagaggacaagaaggctccacgcatttgacttttaagacttggtgtttggttcttcattcttttactcagatttacgccttacaaagggaatctctggtccagacacttgggcctggcaagggtggctgatggtggttaggctgcacacttgagaagcaaacaggagcagggacgtcctgccacacaggcacgcacagggtcagcctctggacacttggcttgggctgcctggcctggggggctagactctggcatctggctgggcacacccaccagtgtttctgtgctctgccgcctgtgagctaccactttcctaaatagaaaatggcattatggggttggggatttagctcagtggtagagcccttgcctaggaagcgcaaggccctgggttcggtccccagctccggaaaaaaaaaaaagac

SEQ ID NO: 16 - DNA, 1899 bp, Rattus norvegicus
gactttgggctcaggatggtggcagcagctatgttgctacggtcctgtccagtgctctctaagggccccacaggcctcctaggcaaagtggctaaaacttaccagttcctatttggtattggacgctgccccatcctggccactcaaggaccaacctgttctcaaatccatcttaaggcaaccaaggctggagcagactctccgtcttggactaagagccattgtcctttcatgctgtcagaactccaagacaggaagagcaagattgtgcagagggcagctccagaagtccaagaggatgtcaagactttcaagacagacctgctgagcttcatggagtcaactacccgaagccaatcagtgcctcgtttccaggatccagagcagactggaggggcacctcccctgctgattcagaataatatgactggaagccaggcttttggttatgaccaattttttagagacaagatcatggaaaagaagcaggaccacacataccgtgtgttcaagactgtgaatcgctgggctaatgcctacccctttgcccaacacttctccgaggcatctatggactcaaaggatgtttctgtctggtgcagtaatgactatttgggcataagcaggcaccctcgtgtcttgcaggccatagaggagaccctgaagaatcatggagctggagctgggggcactcgcaatatctcaggtacaagcaagtttcatgtggagcttgaacaggagctggctgaactacatcataaggactcggctctgctcttctcctcctgttttgtggccaatgattctactctctttacactggccaagcttctgccagggtgtgagatctactcagacgcaggcaatcatgcctccatgatccaaggcattcgtaacagtggtgcagtcaagtttgtcttcagacacaatgacccaggccacctgaagaaacttcttgagaagtccgatcccaagacaccaaaaattgtggcttttgagactgttcactccatggatggtgccatctgtcctctggaggaattgtgtgatgtggcccaccagtacggggccctgaccttcgtggatgaagtccatgccgtaggactatatggaacccggggtgcagggatcggggagcgtgatggaattatgcacaagcttgacatcatctctggaactcttggcaaggcctttggctgtgtcggtggctatatcgccagcactcgggacttggtggacatggtgcgctcctacgctgcaggcttcatctttaccacttcactgcctcccatggtgctctctggagctctagagtctgtgcgtctactcaagggagaggagggtcaagccctgaggcgtgcacaccagcgcaacgtcaaacatatgcgccagctactaatggacaggggctttcctgttatcccctgtcccagccacatcatccccataagggtgggtaatgcggcactcaacagcaagatctgtgatcttctgctcgccaagcacagcatctatgtgcaggccatcaactacccaactgtgcctcgtggtgaggagctgctgcgcttggccccctccccccaccacagccctcagatgatggaaaactttgtggagaagttgctgctggcctggactgaggtggggttgcccctccaagatgtgtctgtggctgcatgcaacttctgccgtcgtcctgtgcactttgagcttatgagcgagtgggagcgatcctactttgggaacatgggaccccaatatgttactacctatgcctaaggagccagctgccttagatgccaaccccacctgcactccccctggggatggtcttcctcctactctctgctttgctgtgtgcttctagctgacttgattctaaaaataaagagcaacctg

SEQ ID NO: 17 - DNA, 352 bp, Rattus norvegicus
  misc_feature (303)..(303): n is a, c, g, or t
cacgagggtggttttgcagtctgctactactgcggtagagcagcgcagaagagaggagatccgggagacccgcgtgagacagtgaacaaatccgatttaatcttcctctgtctggctgagttgcaggaacctcactgtgcccgggtctcccgcccaagaggagccgttggccgagacccgaggcgtgtactgactgaccgccagctgccatggctgaccacctgatgctcgccgagggctactgcctgctacaggtgccgcacgcgccccggactctgcagccatacccaggccaaggcctanacagcggtctgcggccgcaagcttattccctttagtgagggttaatttt

SEQ ID NO: 18 - DNA, 518 bp, Rattus norvegicus
tttttttttttttttttgactcttgactacaagatgatttattgactaaacaggcttcccagcctgtcctctgcccagtgcagggaagataggttagaccctggcagaaggttagttctagatgagggctctgcagcttgcagaggtgctcagtgcttactcagaggattagtgaggcagctcccaggggttctagctgactccattcttgagtgtctgctccgtgggggtgcaggaaggcagcccctacctcagcaccccgaaagcttgggcataaaaaggtgaaccgtgtctgggcaggttggactaagccctgcctggagccgcagaggtaacaagttgagtggaataggtaggcatggcccctgcagtgactaggccggatggtggaagaggctgcctgtgcgcagccgcctgcagagctcctgccatagcagctgcctctctcgctgagcacccttcatgatgctgctgttctccagggcacggcgggacaggtacgggagcacctcgtgccg

SEQ ID NO: 19 - DNA, 616 bp, Rattus norvegicus
gggaacgctgaacagtaagacctattatactctgcacagacgtttaaccgtggacgaggccacagcctctgtctcagaaggaggaggacttcagggcattaccatgaaggatagtgatgaggaagaagaaggctgaggacgcacagttcctcaagaggaaggaaggcctcggcagcgttttgtgcaccttgtctttgtagctgtatttactgtcttgaccaaataaaaatgctctaatttctttagaaagcgaacttggagataaagagagaaatgccatgtatttttggaaagaaaaccatatttcatatgtatagcttcacctagaacttggctgtgctcgtgactggacgatgagctacttttgttgttttgttttttaattctgttttcttttgtttgttgttgttagtatatctgactttgcttttcaatagtcatatcaacataacgtcctatgtggtcctatgagagaagcaccttccatgacacagcattgtgaagagaagccatttctggtcacagcgccaaccctactgtacttccgtcaaagtggtgtgccttccagttaccatttatgtacaaagtttgttctgtaatttgccagtgttgtgat

SEQ ID NO: 20 - DNA, 630 bp, Rattus norvegicus
gtctaatgtcagggcgaaatcaagcccacggcaaagaattatgagacatccccaggcaccaggctcacactcccagggcaggaccaaagactgatgcctagagcgggtaaggggtgtcgtgggtgtccctgagaagctcagtccagagggcctttgtctaagagactctgagaaagggatgggtggcaggaagcttggggaataagggtattaagaagagaataaattaaagggggggcttgagggacaaggggcctgtgctgtccttcaaacagctgggagcagaccaggggtgggaaagagggtggcgggaagagcttgatacactatcttaagaaacaccgtttacccacttccctcttaaccactgcagtgcacaacgagccagggcacagggcaggagcccacatgccccagtggctttcaacatggcatgggtataaagggaagcagctgagggatatctcaggaaggggaagttatcccctggtccccaaatgctataaggcacaattcttggaggcaactagattccatccaaaatattaaaggaaaaaaaaaaaacaacttcaaaacagaaaactttagatcccaggtctactgtgacttcgcttgggcctggtcaacact

SEQ ID NO: 21 - DNA, 564 bp, Rattus norvegicus
caaagtacaagatgatgtcacaaaactggatttgagattgaaaatttcaaaagaggagaaaaatctgggtttatttatagttaaaaacaggaaagatttgattaaagcaacagatagttcagaaccattgaaaccatatcaagactttgttatagatgtaagtatgtgttaagtttgatcagaaaatagttctcactggagagaagtaattttttaaactgtagatttgatgtaagtatttaaatagtagaatgaagttgtaagtgcttagtatgtgataaaactgtgtcacttagtctagggaaccggatgcaactgcccgtgtttgtgagctgctgaagtaccaaggagagcatgctcttctcaaggagatgcagcagtggtctgtgccgccattccctgtgagtggacacgacatcagaaaagggggcatttcttcagggaaggaaattggggccctgttacagcagttgcgtgaacagtggaagaaagtggttaccaaatggaaaaagatgaacttctaagttacataaggaagaccttgaaaacgaactccgtatgaaa

SEQ ID NO: 22 - DNA, 584 bp, Rattus norvegicus
gcacgctctgtaggccctgggctcggatgtttgctgtctgtgatcctgtgctaagttcactgtgtgtctccagggtctgcctcccaattctgccctgcaccttgcaccttccaaagtaggagagaacctaaaccacacagaggttactgcgtgcgtgcgtgcgtgcgtgcgtgcgtgcgtgcgtgcgtgtgtgtgtgtgtgtgtgtgcgtgcgcgcgcgcgcagctgtgctttcttgcgtgagtttaagacttccccaaggcatactcttccccaaggcttgttagtttggccgttgacaccgtcttgatggttttatttacaacgaatgttcttattttcattcactcataagaaagcttgctgtgcgtcattattcaggatcccagaaagggacctttttcttgggtttccttcacaatgccacatgacgcgacataggaaaccattgctcactggagcagatatgcccaggaaccaccagacacctcttctgtagagaaagctttcagcaaaggtcaggccagagagaacttaggacaataaactgcgtgtctggttttgggttttgaaacctgtgggaag

SEQ ID NO: 23 - DNA, 548 bp, Rattus norvegicus
ttttgctctctggaatgcgtcctggcttaggctgtctcggccctgccggacccatttaccgcggtgccatgccctctctttccgcgcgtggaagcttgagatgggtcactgatgacgacctagacaaactttgcaaagcaggttgccacctgttggaataaccgctccacccccagccgaaatgcaagtctgaacaatggcgggtttgagtggctcaagggcccacaattcttcactttaaaactgatgcactcataagagtatttcctaacatgccccacattcagagacctctgctcaccacctcagcaatgcccagggaggttcgtctggcgtgggcaggctctcccttgaaaggatctgacttagttctactttgcactggggcgaaatgctaatttattattattattattactactattactattatcttggagagacaaggtctctctggcgatcctggaactccccaagttaaccaggctttgacacccaaattctgggattaaagctgggccccaccgcgccacactgtagtgaatttt

SEQ ID NO: 24 - DNA, 370 bp, Rattus norvegicus
tttttttttttttttttgggatgtaccaaggcagagactcctgagaagtctgtttataaagtcacaccttaacagagagtgcgggtcaggctcaaaggatggtacacttggccttctccacccaagggttgttcttcatcaagtccgggttcaagaaggggtccttggggatcccgtcctcaatccacttgaggagctcggggatggtcttggatgacatctccctcttgaaggccagttggtacttgtggctctccacctccttcttcatctggggcacatcccactcctccatggcgtctggggcgtcggaggtagccagcctgaggttttgaaggagacagcaaaagcagagatgaccctcgtgccg

SEQ ID NO: 25 - DNA, 516 bp, Rattus norvegicus
tttttttttttttttttactttttccataatagcctttaatactaaaatgcattaaactttttgaagcagagaaaacacatttaagaggaaaacagagaaccaccagataaaaggaaacaatgtggcaggtaacaccatttaaaacagaactgtaataatttaataaagctgctttatcatcttcataaaatagacacagtggtgattctttttttgtctttttcgtttttacaatgagtcaatcactgtgaaaatgtacataacatacagatcagcatatacaaccatttcatcttcatataatcagcaacagagactgcttaaagagacatactgtacttgcatccttctacatcgccaccctggtttgacaggtaaacagtaatagtgtcatcaggctcattcccctcactagaggagcgaaggcgtaagccttttgcaactaataccttagcattcaaatagcacaaggagggatttgttgctgaatttctagctagacctagtctggctct

SEQ ID NO: 26 - DNA, 707 bp, Rattus norvegicus
gaactgaaaaaatattggcaatgttttattagtatgagtgtgcctcagagttcacaccacagtgttgatcctacatgagttcaatgttgcgctatacacagccaatgctggacaactgtgatgggctacctcctgctgttgaacctctccgacatcatcctctcacatcgggaggcagaagattcccaaggtaatggcagagtgcttgcttcactcttaggctattctgatgttacaatcaagccacgactcttactctggctgatccctctcaggtgtcagagagctgttctttcccaaatcctcaggtatgggcaaatcatgagtcgtttttcctctacacattaagcttccacagcaattcctcctgaattcttctgtagtcaagtcactaaattgatttaggcccatgtagaagctggtcttgccctgcttatagtctgcattgtgcgcctcaattgtcttcttactctcctcccacactgctcttctgtgtcgttcttcatccgggctgtaggtttttccaaatttcttcttccattcctcccactcggtatccaaacttggatcaggagatggagcagctgaggccattcccaagcagaagacgggcagggagtcagcactaattgggacctcaaaaccttgcttgtctctgctcccacttgaagtgctgcaacttctgttcacagatggaaaccagaaat

SEQ ID NO: 27 - DNA, 627 bp, Rattus norvegicus
ccaagaaaataaatttattgaactttgaggggaaaaatccacagacataaaagaaagttaaatacagactgcaggacacaactagtcacatggtgaataatggcttgtgtggccagcaaagtaccaaaaatgacattctgggactgattgaggtatttagctatttttggctatagcaacgtggtcagcctatggtgaaatggtaagatagttcatcaaaacacacatctttaagctgaataggttctaagttgatgatacttcatatgaactaaatcatgtagccattggggaaaccatagcagcaaggtagcagaaaaaaatctattaaaatctactacagaataacacagtgaaccttaaacacccaagtctaaatttttcactgtctctcctgcatgaaagagatttaaaaaccactaaacattaagcctgtttccccacaaaggtctctgcaaaatggtaaatatcattgaaagttaaaggtaattttaaagtttcaagcatctaaaaagtgcttaaaatgagaagtattctttagttggctgaaagtcctccttctctcataaatcttttggggggctaacccaaaaagctgtgtctatgttattacataattaaagggga

SEQ ID NO: 28 - DNA, 586 bp, Rattus norvegicus
gcagaaacagcatccatggatgagatgactgcttgtattttagtcagggcttagaagtttagacagtcgatttatattgcttagtgatggaaagttggttttttttttttccttccttcaatattaaaaaaaggctctgtatgcatggtggggctatgtacatactcggtactgagccctgttaatctcacaagtgttatttatggttcaagctttgtaaactgtataaatgtaaaaaacaaaccccacacatacctctggagtatatggtaaaaccgaggagaagctatgaggcgaccttccccgtgcaggggggagcggtctcagcaggtggtaacgtccacacaaggggctttctgtggcagtctgtgtagacttactatgtacagagaggttatttcttctgccccttttccttccaacttgttgttggcctttaaaagcaattttgaaaactgggtttccagttgcggttttcttttgagatttttcaggaagaatgagccggagtactcccaagttcatgagactctggttgaatggagagccatgtcagatttgaactaacgagcgggtagtttggtct

SEQ ID NO: 29 - DNA, 689 bp, Rattus norvegicus
gatctaagcctgaggaaataaaagtatcaaacactgaatgccatggtactaccataaagaaataacagaaaacaaattacatttaaacagaaattggggcatactgctacttttatgaccacttgctgatttcaattatcataaattcatctgtgggaagttgtgaaagcattaccaatgagagataaaatgacttgtgtttgcacaggtgaaaatttgtcacccagctttcataattgtgaccagtgacctgtacagaccatcatttgtgaaaaattagtcatgctcagtttatttcttcatgttatctagggacctgtatttctaggtgatgtgctcgttattttcattatttacttatttttaaaccaacgagaaccactggattgtggtctttatctaaattcaagagaccggtaactcaaaaatcagaaataatttgagttatataggttaagaacatttttttttagcctgcaagctattaataatgacactaccccctaaatgaaactaatatgaaaaattttaatatgactaaatgacttatataatgaaataattaccggtttatacgaaccagaatgttctcgtcctgaaaattctattcccacttcctgcaaggcaccacttagtcctttgggttttctgttataaaaaagctaatgaaataaaataaagatattttt

SEQ ID NO: 30 - DNA, 531 bp, Rattus norvegicus
tttttttttttttttttgctaggtaggcccaggccagccagccttgaacttactctgtgtagcactggcttacattgaatttgtagaaatcctcctacttcagcttcacaagtactgggacgacaagtgtttaacactatatctatctttctaaattttttattgtttttatgtatgtgggtgttttgcctgcatttatgttttgtcaccatgtgtgtgtctgaaaactgaattggcagaagggttagcagtggttgtttctgtttttatggccctggtggcacagtggctgactaaaactggggtaactattgctagaagaagaaaaaggacagagcacacagtatgcaggtggaaagtgagggagatactcaagaagtggtatgttggggtgatgtgtatatattctgagtcttgtcaccactgaactgggcatgtttacagcagcctccaaggaatgcacctaaggccaaaagccacggatctcagttgcagctcaagatcaagggagtgttaagttcagccaagtta

SEQ ID NO: 31 - DNA, 558 bp, Rattus norvegicus
ttctatgacaaaggcagcattccatttaaaagtgtattcacgtacaaagtattaatgctaaacttttagcagaagatgaagtaaaacatggtattctgtgaggtgcaacacacattgacttattttatatttgacaacatgaattttcacttgaaatatgagtaaaattactggagaaacaaaacatacattgcctgattctcttgaccatagaaagtacgtactgctcctctgaacacaacatctgatcgctatcacagaagtctttatcttcttatctgcaatccttttctcagtttctgtatcacaggagcacgtctttctcacccattccacagcatccggtgctaaccgaagcagactcctgccaggaagtagcccctcccttctagccggagcaagagtccatctttcagtgccgtaactgcattattcagcactcctcttgctctaggaaagctgttttgatctccctttattttaaacaaattttaacatcttgtgtctgacaggctagagtcaggatggggctttacatagatgtaaacgaatggaatc

SEQ ID NO: 32 - DNA, 630 bp, Rattus norvegicus
gccactttctgacccgactttattaagggatctctttcctctcctctcctatctagtgcttgggggtagatgggggcacaaagaagagggatggagaagggtgggaaaaagaaccccagcaccaggtctcatgcctgcacgccaaaagtttagccatggagtcatctgattccagtggactttgaaggttgttaccccaaaatttcggatcctggtgatcaattctgacctcttctgaagaacatggttggtggagtttgttaccttttgttccactttcggaatgtcctcactgcctgttcattacgtctgtccgtttcagtttgtacttcagcaatgggtcaattgcttcttgctcttctttttctccttcccaggccggagaggcacacttgtcctccagcttcagtgtagcaaagggtctgggattcttgaccggaggcagatcccagatcactgtccttcccagagcctccttcatccactgattccaacatggaaaccaatgtcatttatgttgtcgttggagccgtctctgctgtggccattattggagctgtgattattggagctgtggtggctgctaagaatccaggtaggaaagaggacgtctgggtgccctgtcagaag

SEQ ID NO: 33 - DNA, 542 bp, Rattus norvegicus
ggaagtgatcagactttggtttattgtaaaacttagcaaagtgtttcatataatccctgacccctcactctgaaaacaaaagcagaaacaattattgcttattttccccctctactttgtctgtgctactgtaagagaagggagaaagattattacaataaataaaaattgagatgtaacagagaaaaataaatcagtctactgagaagtattaggagcaacagaaatttcattaagcagtttaaaaataagcttctttaaaaaggttgccttattaaaataaatcacaccaaaaatatagcagcagagaagaaggatacatacaagttaattgcacatcagtcccatgcaaaaacgtggatcattagccaaagcagtagtactcagaatccagcttgggatgcttgtgcagagcttgagagtcctctatgatagagctgtcactgaactgatccaagtctgaaggggtctgatggcctggtacatcatctgccaaagtgtccaggtagctccctaccgagatgccatccagcccatcactg

SEQ ID NO: 34 - DNA, 680 bp, Rattus norvegicus
  misc_feature (528)..(528): n is a, c, g, or t
aatactcacaagatagcctctgctttaataataaagatctttgatactatgtctttaaatttctgctagcttctatttaaccgctctttcagaagttatttatacatatttactcaacacattctggaaaatataagaactaatgtcgattctcaaggtcacacacggtggcaggttacaggagcacactgactccttgttgcatataagttgcatctaagaaacatggcgggcacattttagtgatccatgcagagtaggttcacgaaaacaaacaaacaaacaaaaccccaaacaattaaacaatagtcagccatgaaaacttaacactcccagtcacgacaggcataacagcaaaaacaccagcactgacggctttgtccaaaccatgctgggcttagtcaattctgcttagaggggccagaaaggacactggcaaccaggaatgttcctgattctcaacactgggttcagactaaactctcctcacatggcttttccctatactgcaggcatttcaggcttcanataaaaacaatcaaccttcactgaaaagttgaaaattgtctcttttgagtctctggaagcagctcgctgtaagtccataaggcaaatgccagggctcaaacaaaagcagcctcgagatgtaaacaataatattaccgccaaaggaattacaa

SEQ ID NO: 35 - DNA, 646 bp, Rattus norvegicus
  misc_feature (550)..(550): n is a, c, g, or t
gcggccgcgcacacacagagacacgctcacacacactcacacattctcagcctggcgcgcgcgcactcacacttgcacaagcactcccagaaccgcaccgggaggaagaaacagtactggtcccactcaagatccggtccggaagcccaccagcccaccggcaaagagagcagtcgctttctcacatagccccttgaccacgtttccttcaagttctcctgcagtcaaatccttggaattcaaaagctggtgacatttcgctggggagtaagggagtcctgaaagggctatggaagcgtcggggattcgttaagcagtttgaaaagcaatcctcattaccttgtttgtacagtaaaccagctcggagtgctgaagtttggcttcaaaggttgtggacagacacattcatattcctcggaccaaaacaggaaagtgaagatgcaggaaaataatacagctgcaagtccctctcttgaccttgtgatgtacctggaagctggttaatacagccactttccaagcctctggtttaagtgatccatacacctgngttcatagcccatcatcttcccagactccagccaggaaaaccggtctgggtgtcctccaagctctgggagaacctgngcatggaggggcatctctg

SEQ ID NO: 36 - DNA, 427 bp, Rattus norvegicus
cacgaggatgagatcctggaggaaaacctgctacctttcaccattatcccaaacccactggctgggagggaggtggccagagaaggttccagtgaggaggagagccgcgaggtcacaggtcagcaggatgcccaggagtatgagaactaccagccagggtccctgtgtggctactgttctttctgcaaccgatgtagtgaatgtgaaagctgccactgtgatgagaagaacatgggggaacactgtgaccagtgtcagcactgccaattctgctacctctgcccgctggtctgtgacacactctgcactccaggaagctacgttgactatttctcctcctctgtgtatcaagctgtggctgacatgctggagacgcaagagccctgatctggccacctggcaagagccgcatttatttctctgaata

SEQ ID NO: 37 - DNA, 533 bp, Rattus norvegicus
  misc_feature (376)..(376): n is a, c, g, or t
tttttttttttttttttggtggtttggaattcttttctcttttgttaaaagaggggtaggaaatggggaccaggtacccctgggctctgggaaacaggcatgcagggaacccttgcaggcaggggctgggtagaagagtcctggagtttcccataatccttcgcaggaaacagcaatgctggcagataaggaggtggagtgaggcagggcccttcaaacaacagggtggcgggccaaggggcttggggctcactctaacatgcaaagtccagctgccccataaactaggttgcttttgaagagcgacatacgtataaatacataagacacagctacacgcacacatgcggagaaggctctgcattcccaagggtanggatctaggcctactggccccaagacaggagtcatcatgtgtctgccaccaagtgattctctgaaacactccaggtggtggggccaggcaggtaagtcttcgttgggatggctgcttggtctccaaggtgctgcccactaggcacccaagccacttt